(19) United States
(12) Patent Application Publication
(10) Pub. No.: US 2003/0161841 A1
(43) Pub. Date: Aug. 28, 2003

(54) Preventive and therapeutic agents for microbe-related syndromes including HIV

(76) Inventor: Kazuo Sakuma, Hokkaidou (JP)

Correspondence Address: Kazuo Sakuma, 2119-1, Kaminayoro, Shimokawa-Chou, Kamikawa-gun, Hokkaidou, 098-1216, JP

(21) Appl. No.: 10/066,224

(22) Filed: Feb. 1, 2002

(51) Int. Cl. A61K 35/78

(52) U.S. Cl. 424/195.15

(57) ABSTRACT

Preventive and therapeutic agents against the HIV virus and pathogenic bacteria, comprising the active ingredients obtained from hyphae of Kabanoanatake [scientific name: Fuscoia obliqua (Fr.) Aoshima].

The said active ingredients have potent inhibitory effects and therapeutic effects on HIV, even in very small amounts. The said active ingredients also have potent inhibitory effects on Bacillus coli, and other pathogenic bacteria.

The present invention offers culture methods which enable the supply of active ingredients having potent anti-HIV activity and other physiological activity, by artificial culture of such Kabanoanatake on a large scale.

### BACKGROUND OF THE INVENTION

1. Field of Invention

The present invention relates to preventive and therapeutic agents for microbe-related syndromes including HIV (Human Immunodeficiency Virus), more particularly, it relates to preventive and therapeutic agents containing extracts from certain mushroom as active ingredients and having preventive and therapeutic effects on syndromes possibly caused by a species of retroviruses, HIV, ALT (adult T-cell leukemia) and pathogenic bacteria. For further detail, the mushroom used in the present invention is a specific mushroom which belongs to the class Basidiomycetes, namely Kabanoanatake [scientific name: Fuscoporia obliqua (Fr.) Aoshima], the mycelium extracts thereof constitute the major active ingredients of the preventive and therapeutic agents of the present invention. The present invention also concerns the procedures to culture hyphae of this mushroom artificially and obtain the active ingredients from it.

2. Description of Related Art

Recently, AZT, DDI and other agents are known as a species of retroviruses, HIV (AIDS virus) inhibitors. However, these chemical agents have problems such as side effects on the human body as well as HIV drug resistance, mushroom extract has been utilized as anti-HIV agents to avoid such problems. There is a published document (1), as an example, of Japanese patent documents before examination, Showa Era 63-316734. This document contains a description of ‘anti-HIV agents’ of which the active ingredients are polysaccharides or protein polysaccharides extracted from Basidiomycetes, but the experiment describes only three kinds of samples; Ganoderma lucidum, Flammulina velutipes Auricularia fuscosuccinea as the main examples of Basidiomycetes. As an example (2) of published Japanese patent documents before examination, there is also a Heisei Era 2-134325, in which an HIV therapeutic agent comprising one constituent extracted from mycelium cultures of Basidiomycetes is published under the title, “HIV therapeutic agent and production method thereof”. In the public example (2), as the range to obtain Basidiomycetes, Lentinus edodes, Coriolus Versicolor, Pleurotus ostreatus, Flammulina velutipes, Ganoderma lucidum, Grifola frondosa are listed, but the embodiment described in it is limited to Lentinus edodes.

Looking through these published examples, in published example (1), 10 g of freeze-dried extracts of Ganoderma lucidum, Flammulina velutipes, Auricularia fuscosuccinea was respectively dissolved in 100 mg of distilled water to be used at a concentration of 1 mg/ml, and in published example (2), brown powder, freeze-dried extract from Lentinus edodes was investigated at concentrations of 0.1, 0.25, 0.5 mg/ml, but actually suppressive effect on syncytium formation was observed only in relatively high concentration of 0.5 mg/ml. This indicates that, in short, extracts from the mycelium of mushrooms of the well known daily such as Lentinus edodes, Ganoderma lucidum, Flammulina velutipes, Auricularia fuscosuccinea have anti-HIV effects, but it doesn't have a sufficient degree of activity. In proof of that, in Case 1 of clinical tests of the published example (2) ((9) clinical test for HIV patients of the published example (1), there is a description of a patient that T4 cell count increased from 1250/mm³ to 2542/mm³, but patients with an advanced HIV disease usually have a lower than normal T4 cell count (i.e. 300/mm³). On the contrary, the fact a patient who has a T4 cell count of 1250/mm³ recovered only leads to a conclusion that anti-HIV activity of the active ingredients (Lentinus edodes) is low. In fact, Case 2 of the same published example describes a patient with T4 cell count of 822 died from complications of pneumonia, in spite of being orally administered the active ingredients of Lentinus edodes.

Furthermore, there is no prior research to suggest active substances derived from mushrooms being valid not only to HIV but also to pathogenic bacteria.

Usually, antibiotics have been used as a remedy against pathogenic bacteria, which had some effects on the bacteria, but due to cumulative use, drug-resistant strains are increasing which causes new problems. For instance, though E.coli bacteria, an indigenous strain, is generally said not to have pathogenicity, among them there appeared E.coli bacteria which can cause severe disease such as Escherichia coli O157, which has recently become a topic of widespread concern and can be treated significantly by conventional therapy. In these circumstances, the advent is anticipated of new anti-bacterial agents and health food ingredients which take place of the conventional antibiotics or incorporate with them.

The inventor has found so far that the extracts from Kabanoanatake [scientific name: Fuscopia oblique (Fr.) Aoshima] has anti-tumor and anti-HIV effects and filed patent documents to the Japanese Patent Office (Tokugan Hei 1-114665 ‘anti-tumor agents and anticancer food that may be administered orally and production method for mycelium’, which was afterwards awarded a Japanese patent, No. 114665) and Tokugan Hei 8-23208, ‘anti-HIV agents’ (‘Tokugan’ mean a patent application. ‘Hei’ is the abbreviation of (Heisei) which is the current Emperor's era that started in 1989). From having sought further benefits on the basis of these observations, it was confirmed that the Kabanoanatake extracts contain great quantities of LPS (Lipopolysaccharide) and pseudo-humic acid (humin like polyphenol complex) which has a high content of methoxyl groups including also monolignin. From there, it was found that the Kabanoanatake extracts are effective in preventing cancers, microbe-related to syndromes such as HIV, ATL (adult T cell leukemia virus) and pathogenic bacteria, especially Escherichia coli O0157.

### SUMMARY OF THE INVENTION

The present invention provides preventive agents effective in preventing various syndromes caused by microbes by killing HIV and bacteria such as enteropathogenic Escherichia coli, particularly Escherichia coli O157 and MRSA (methicillin resistant Staphlococcus aureus, or at least inhibiting the growth of them, wherein the active ingredients thereof are the extracts from Kabanoanatake [Fuscoporia obliqua (Fr.) Aoshima].

Kabanoanatake belongs to Basidiomycotina, Hymenomycetidae, Aphyllophorales, Hymenochaetaceae, Kabanoanatake [scientific name: Fuscoporia obliqua (Fr.) Aoshima], and it is classified as growing naturally on trunks of birch trees such as white birch (Betula platyphylla Sukatchev. var. japonica) and Betula ermanii, which forms black sclerotia like a coal-like mass. Its sclerotia may grow up to about 20 cm long, originally known as evil fungi (form of cancer) in regard to birch trees such as white birch. However, the inventor (Kazuo Sakuma) embarked on the research by paying attention to Kabanoanatake (common name: Chaga) having a strong life force, whereby it has been found that the hyphae extracts thereof may have anti-cancer effects and possesses inhibitory effects against HIV, enteropathogenic Escherichia coli, especially Escherichia coli O157, Klebsiella pneumoniae, Pseudomonas aeruginosa, MRSA (methicillin resistant Staphlococcus aureus), clostridia of gas gangrene.

Through the present invention, it was shown that natural and cultured Kabanoanatake are both equally effective. However, it is preferable to make use of Kabanoanatake grown in artificial cultures when required in large amounts, since natural Kabanoanatake cannot be taken easily and its yield is low. There are several methods for artificial cultures, but preferably sawdust culture and liquid culture are used. Furthermore, hyphae and its secretions were proliferated artificially by planting Kabanoanatake hyphae on the green wood of birch trees, or formed sclerotia by growing this hyphae so it can be utilized effectively in the present invention. Kabanoanatake inoculated hyphae artificially are in line with the natural form.

The method of liquid culture of the present invention is described here, consisting of inoculating the seed fungi from precultured Kabanoanatake in a liquid medium (culture solution) containing a mixture of a carbon source selected form the group of malt, glucose, saccharose and starch as well as peptone and yeast extract with water and phosphate buffer, incubating at a temperature of around 20 to 30° C., preferably at 25° C., for a short period of 20 to 49 days, or in some cases for a long period over 100 days, and growing the hyphae by shake culture method. For liquid medium of the present invention, suitably adding wood constituents, to be more specific, one ore more substances selected from lignin sulfonic acid, lignosulfonic acid sodium salt, lignosulfonic acid sodium salt acetate, lignin alkali, lignin organosolv, lignin organosolv acetate, 2-hydroxypropyl ether, lignin hydrolytic, hydroxymethyl derivative, lignin organosolv propionate, betulin (betulinic acid), or lignin salts, the growth state, yield and potency of hyphae can be improved. The growth state of hyphae is determined based on the density of the black pigments developed in hyphae and in the culture medium, nutrients in the culture medium, particularly decrement of carbon source, protein quantity secreted by hyphae in the culture medium, and pH of the culture medium.

The harvest (collection of the active ingredients) is done by determining the time of formation of the active ingredients having sufficient anti-HIV activity, which was produced by hyphae. One of the practical harvest methods for the active ingredients by liquid culture method concerning the present invention, is to extract mycelium along with culture solution (including secretions of the hyphae) in hot water, filtrate them, filtrate the filtrates again where necessary, and dry them by vacuum-freeze to get the active ingredients in a powder form. According to the present invention, practically, the large part (about 90%) of the active ingredients having anti-HIV activity, etc. are contained in the culture solution, and the active ingredients extracted from cultured mycelium are relatively small amounts (about 10%). Liquid medium (culture solution) is not only a source of nutrition to grow hyphae, but also a place to store the active ingredients secreted from the growing hyphae.

Whether the active ingredients of Kabanoanatake obtained by the methods of the present invention have anti-HIV activity or not was determined by the following test procedure, which does not constitute the present invention by itself.

(1) Test Procedure

This test to measure cell damage was conducted by using MT-4 cells. 96-well microplates were arranged as eight rows, A-H (sample numbers) by twelve columns, 1-12 (well numbers). 100 μl of dilution (cell culture solution) was placed in each of all the wells, and further 100 μl of sample A was added to well A1 and 100 ml of the solution was thrown out. Then, two-fold serial dilution was carried out in each of the subsequent wells from A2 to A12. Namely, starting from 1:2 dilution in A2 well, the dilution ratio of the sample was serially increased as follows; 1:4 in A3, 1:8 in A4 and finally, 1:2048 in A12. The same serial dilution process was repeated for the samples, B to H, from well B1 to H12. After all the required samples were diluted, 100 ml of dilution of the wells were thrown out. 100 μl of suspension of cultured cells (MT-4 cells) incorporated with HIV was placed into all the wells so that the total liquid amount in each well became 200 μl. The cells had been cultured, and after that the inhibition level for HIV was observed.

(2) Numeration

1) The effective dose for inhibiting HIV is calculated as follows: in the case of inhibition against the HIV virus observed in the well number 8 (at final dilution, 1:128), where the specimen stock solution is counted to be 100 μl÷128=0.78 μl, which is converted into 0.78×10=7.8 82 l per 1 m dilution; The effective dose for inhibition is expressed as 7.8 μl/ml.

2) Amount of cell damage

The samples were diluted in the same manner as the ones in the above inhibition test for HIV to measure the amount of cell damage. In each well, placing cell suspension culture (MT-4) which was not added the HIV virus, and liquid amount where the cells die (dilution ratio) were measured with values represented. This indicates of the safety of the said samples in biological cells.

### BRIEF DESCRIPTION OF DRAWINGS

An embodiment of the present invention will be described below with reference to the accompanying drawings.

FIG. 1 is a graph showing the extracts from the black part of Kabanoanatake which is effective as anti-HIV agents (black circle), Kabanoanatake liquid culture extracts (white circle) and inhibition ratio for syncytium formation (ordinate) by conventional AZT (black triangle) in varied consistency (abscissa), by comparison. When 0.1 μl/ml of the extracted solid content from both the black part of Kabanoanatake and the cultures of Kabanoanatake are used respectively, each solution concentration is 350 ng (nanogram).

FIG. 2 is a graph showing the inhibition ratio for HIV infection (ordinate) in varied consistency (abscissa) regarding the extracts of the black part of natural Kabanoanatake (black circle), extracts by sawdust cultures (white circle), hyphae grown in liquid culture and dried by heating (black quadrangle), viable hyphae grown in liquid culture and viable hyphae dried by heating (white triangle), filtrates grown in liquid culture and dried by heating (black triangle), which is anti-HIV agents concerning the present invention. Each extracted solid material is 350 ng when the concentration level of the solution is 0.1 μl/ml, and it is 3500 ng when the level is 1 μl/ml.

FIG. 3 is a graph illustrating how Kabanoanatake of this invention affects the cell viability within both the non-infectious systems and the infectious systems in varied consistency, with the viability (%) on the ordinate and the elapsed number of days on the abscissa.

FIG. 4 is a graph illustrating the HIV P24 antigen yield (ordinate) of Kabanoanatake in the same consistency as FIG. 3 and the elapsed number of days (abscissa).

FIG. 5-A is a graph illustrating the pretreatment effects on HIV by Kabanoanatake, and;

FIG. 5-B is a graph illustrating the pretreatment effects on target cells by Kabanoanatake.

FIG. 6-A is a graph illustrating the correlation between the pretreatment time of target cells by Kabanoanatake and the infection inhibition efficacy, and;

FIG. 6-B is a graph showing the inhibition ratio when Kabanoanatake was added in various incubation time periods, after 1 hour pretreatment of the cells with Kabanoanatake.

FIG. 7 is a graph illustrating the suppressive effect of anti-HIV agents concerning the present invention on the syncytium formation of non-infected cells that were cultured together with infected cells.

FIG. 8 is a graph illustrating the infection inhibition efficacy in varied consistency concerning the five kinds of anti-HIV agents in the present invention.

FIG. 9 is also a graph illustrating the suppressive effect on syncytium formation in varied consistency concerning the five kinds of anti-HIV agents in the present invention.

FIG. 10 is a list showing the results of the test for HIV.

FIG. 11 is a graph illustrating perfect inhibition effects on HIV, using a strain of Kabanoanatake (AIW ro-4) in liquid culture for 62 days, under extreme conditions of diurnal incubation temperature of 33° C. and night incubation temperature falling to 8° C. to 10° C.

FIG. 12, associated with FIG. 11, is a graph illustrating cell damage (which dose not mean ‘cytotoxicity’), concerning a strain of Kabanoanatake (AIW ro-4) in liquid culture for 62 days, under extreme conditions of diurnal incubation temperature of 33° C. and night incubation temperature falling to 8° C. to 10° C.

FIG. 13 is a graph illustrating perfect inhibition effects on HIV in a culture medium including lignin as wood constituents in a long-term culture test, using strains of Kabanoanatake of AIW-27 and AIW-4, under extreme conditions of diurnal incubation temperature of 33° C. and night incubation temperature falling to 8° C. to 10° C., in addition to restricting the infiltration of oxygen.

FIG. 14, associated with FIG. 13, is a graph illustrating the change in cell damage in culture medium including lignin as wood constituents in a long-term culture test, by means of strains of Kabanoanatake (AIW-27, AIW-4), under extreme conditions of diurnal incubation temperature of 33° C. and night incubation temperature falling to 8° C. to 10° C., in addition to restricting the infiltration of oxygen.

FIG. 15, associated with FIG. 13, is a graph illustrating the change in anti-HIV effects by addition of lignin substance as wood constituents to culture medium, in liquid culture for 34 days, using strain of Kabanoanatake (A-2W-3), under extreme conditions of diurnal incubation temperature of 33° C. and night incubation temperature falling to 8° C. to 10° C.

FIG. 16, associated with FIG. 15, is a graph illustrating cell damage (which does not indicate ‘cytotoxicity’, because constituents of Kabanoanatake are derived from natural products and taken orally) by addition of lignin substance as wood constituents to culture medium, in liquid culture for 34 days, using strain of Kabanoanatake (A-2W-3), under extreme conditions of diurnal incubation temperature of 33° C. and night incubation temperature falling to 8° C. to 10° C.

FIG. 17, associated with FIG. 13, is a graph illustrating the change in black color tone observed at 500 nm in culture medium including lignin as wood constituents in a long-term culture test, using strains of Kabanoanatake of AIW-27 and AIW-4, under extreme conditions of diurnal incubation temperature of 33° C. and night incubation temperature falling to 8° C. to 10° C., in addition to restricting the infiltration of oxygen. (This is considered as the standard for the harvest of Kabanoanatake.)

FIG. 18 is a graph illustrating the change in humic acid in culture medium including lignin as wood constituents in a long-term culture test, using strains of Kabanoanatake of AIW-27 and AIW-4, under extreme conditions of diurnal incubation temperature of 33° C. and night incubation temperature falling to 8° C. to 10° C., in addition to restricting the infiltration of oxygen.

FIG. 19, associated with FIG. 13, is a graph illustrating the change in lignin tannin in culture medium including lignin as wood constituents in a long-term culture test, using strains of Kabanoanatake of AIW-27 and AIW-4, under extreme conditions of diurnal incubation temperature of 33° C. and night incubation temperature falling to 8° C. to 10° C., in addition to restricting the infiltration of oxygen.

FIG. 20 is a graph illustrating the change in protein content in culture medium in a long-term culture test, using strains of Kabanoanatake of AIW-27 and AIW-4 under extreme conditions of diurnal incubation temperature of 33° C. and night incubation temperature falling to 8° C. to 10° C., in addition to restricting the infiltration of oxygen.

FIG. 21 is a graph illustrating the values of perfect (100%) HIV inhibition activity by strains of Kabanoanatake hyphae, A to E, cultured at the ideal temperature for cultures of around 25° C., on the 110^{th} day of liquid culture (lignin substances as wood constituents were not added to the culture medium).

FIG. 22 is a graph illustrating comprehensively the values of perfect (100%) HIV inhibition activity by strains of Kabanoanatake hyphae, A to E, change in protein content, change in color (500 nm), when culture was conducted at the ideal temperature for cultures of around 25° C., on the 110^{th} day of liquid culture (lignin substances as wood constituents were not added to the culture medium).

### DETAILED DESCRIPTION OF THE PREFERRED EMBODIMENT

The description of the microbe-related syndromes including HIV concerning the present invention and therapeutic agents thereof are given below; in regard to (I) embodiment as anti-HIV agents, (II) embodiment as anti-bacterial agents, and furthermore (III) embodiment fully as a liquid culture method.

(I) Embodiment as Anti-HIV Agents

It was found that anti-HIV effects of the extracts from natural Kabanoanatake are more potent in the ones derived from the black part on the surface of sclerotia than in the ones from the non-black part (brown) of the internal part, which could prevent, at even 35 ng, the cell fusion (which causes syncytium formation) induced by HIV-infected MOLT-4 cells. In the results of analysis conducted in the same manner regarding the extracts obtained by sawdust culture aimed for mass production of Kabanoanatake, it was also seen that 60 to 70% of extracts from natural Kabanoanatake have anti-HIV effects, under certain conditions. Furthermore, as a result of the analysis of hyphae grown in liquid culture and dried by heating (dried at 105° C.) and a cultured filtrate, using HIV-BRU (100TCID_{5 0}) used PHA-stimulated normal human peripheral blood mononuclear cells, significant anti-HIV effects were revealed in the hyphae grown in liquid culture and dried by heating. ED50 (50% inhibition activity) in the inhibition of the syncytium formation of the said natural Kabanoanatake was 35 ng/ml (equivalent to 0.01 μl/ml). These facts show that Kabanoanatake has anti-HIV activity at very low concentration, which cannot be attained by the mushrooms of the two published examples cited above. Remarkably, that the activity was found in a state where the active ingredients are not isolated. It also found that Kabanoanatake extracts of the present invention coupled with ingredients of herbal medicine work synergistically, whereby the effects of anti-HIV agents are enhanced and established.

In the present invention, by using natural Kabanoanatake, the extracts obtained from sawdust-cultured Kabanoanatake, hyphae cultured in liquid and dried by heating, cultured viable hyphae, and a cultured filtrate, anti-HIV activity was examined in respect to two forms of the so-called growth of the HIV; the syncytium formation and infection. Detailed account of which is given below.

Test Samples

1. The black part of Kabanoanatake (natural)

2. Extracts of cultured Kabanoanatake (sawdust culture)

3. Hyphae of cultured Kabanoanatake dried by heating (liquid culture)

4. Viable hyphae of cultured Kabanoanatake (liquid culture)

5. A filtrate of cultured Kabanoanatake (liquid culture)

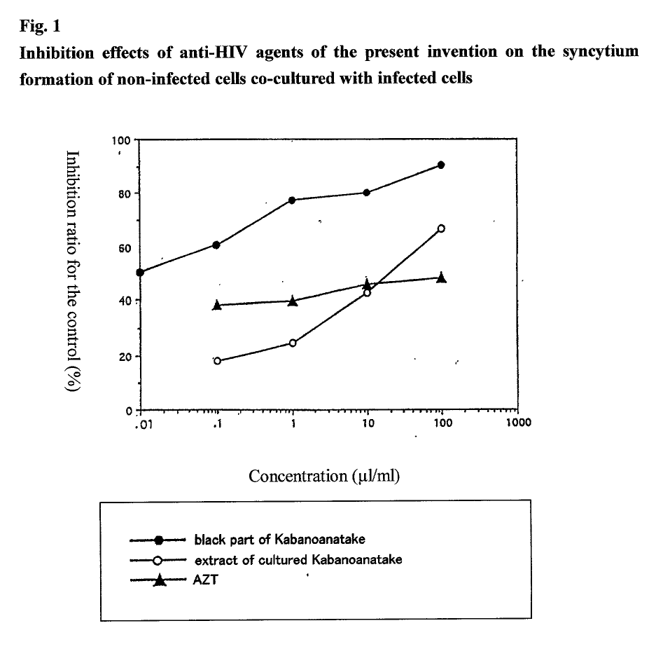

(1) A Test Regarding the Inhibition of Syncytium Formation (Fusion Assay) (FIG. 1)

When Molt 4/c18 cells (non-infected cells) and Molt 4/HIV IIIB cells (infected cells) are cultured together (co-culture) at the proportion rate of 1:1, then non-infected cells adhere to the infected cells, which causes cell fusion and syncytium formation, one form of the growth of HIV. When the addition of the black part of natural Kabanoanatake (sample 1) and extracts of cultured Kabanoanatake (sawdust culture) (sample 2) were added to this co-culture system, the suppressive effect on syncytium formation was recognized.

Test Procedure

Molt 4/c 18 cells (1×10⁶) plus Molt 4/HIVIIIB cells (×10⁶), the said sample 1 and sample 2 were placed in each well of 96-well microplates respectively, in increments of 100, 10, 1, 0.1 μl/ml, and incubated for 24 hours. After that, the diameters of the cells thereof were measured by a multilizer The cells of which the diameters are more than 20 μm were regarded as huge cells, and the appearance rates thereof were weighed. The results are shown in the graph of FIG. 1, with the inhibition ratio for syncytium formation (%) to control on the ordinate and concentration of the samples (μl/ml) on the abscissa. The black part of Kabanoanatake demonstrated more than two times inhibition ratio of as strong as AZT. The extracts of cultured Kabanoanatake at the concentration of more than 10 μl/ml also showed the inhibition ratio that was superior to AZT. Furthermore, it is striking that in the graph of FIG. 1 the black part of natural Kabanoanatake of the present invention showed efficient effects assessed as ED50, at the concentration of 0.01 μl/ml (equivalent to 35 ng/ml).

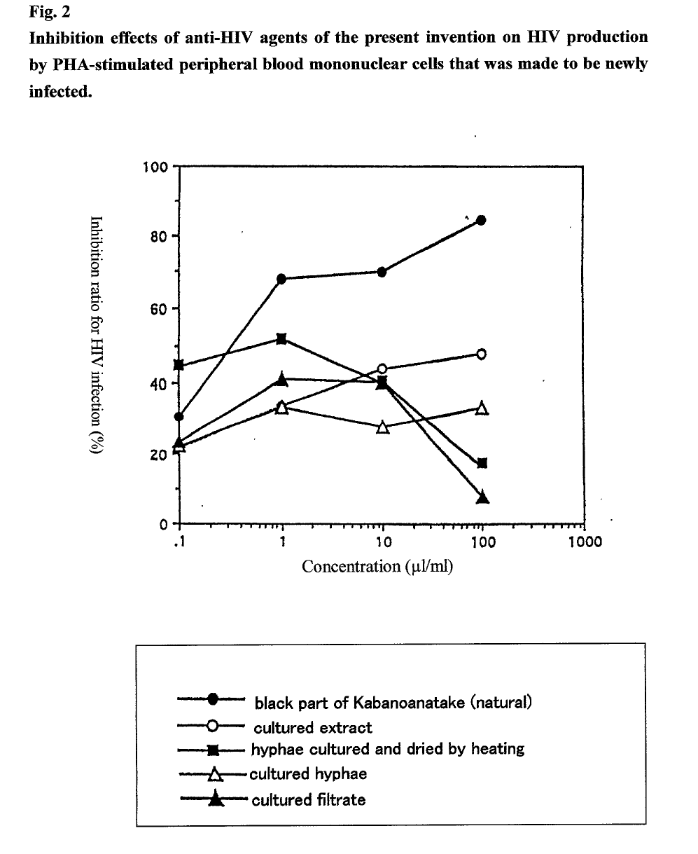

(2) Neutralization Assay (FIG. 2)

The other form of the growth of HIV is the so-called infection where a virus jump out of one cell and get into a healthy cell. It was examined whether the active ingredients of the present invention could inhibit the infection, with the use of the said samples 1 to 5.

Test Procedure

After pretreated stimulated with PHA-blast (PHA [phytohemagglutinin]—stimulated normal human peripheral blood mononuclear cells [PBMC]) (3×10⁶) and samples 1 to 5, at the concentration of 100, 10, 1, 0.1,0.01 μl/ml and at the temperature of 37° C. for 1 hour, HIV-BRU (0.03 cpm/cell) was added, incubated for 24 hours, washed and further incubated for 5 days. After cell viability was identified and the HIV P24 antigen was measured by ELISA method, the infection inhibition efficacy was studied comparatively. The results were given in the graph of FIG. 2. The graph shows the inhibition ratio of infection (ordinate, %), and the concentration of the samples μl/ml (abscissa). High inhibition ratio was observed in the extracts of the black part of natural Kabanoanatake (black circle), particularly it was 70% to 90% at the concentration of more than 1 μl/ml (3500 ng/ml). The cultured extracts (white circle) was obtained by sawdust culture. Hyphae cultured and dried by heating (black quadrangle), cultured viable hyphae (white triangle), a cultured filtrate (black triangle) are all hyphae obtained by liquid culture method, and hyphae dried by heating indicates of the hyphae cultured in liquid and dried by heating at 105° C. (which smells more spicy than the hyphae obtained by other dry techniques) and cultured viable cells indicates of the hyphae liquid cultured and then freeze-dried after boiled in hot water. Hyphae dried by heating (black quadrangle) showed comparatively high inhibition ratio at the concentration of 0.1 μl/ml.

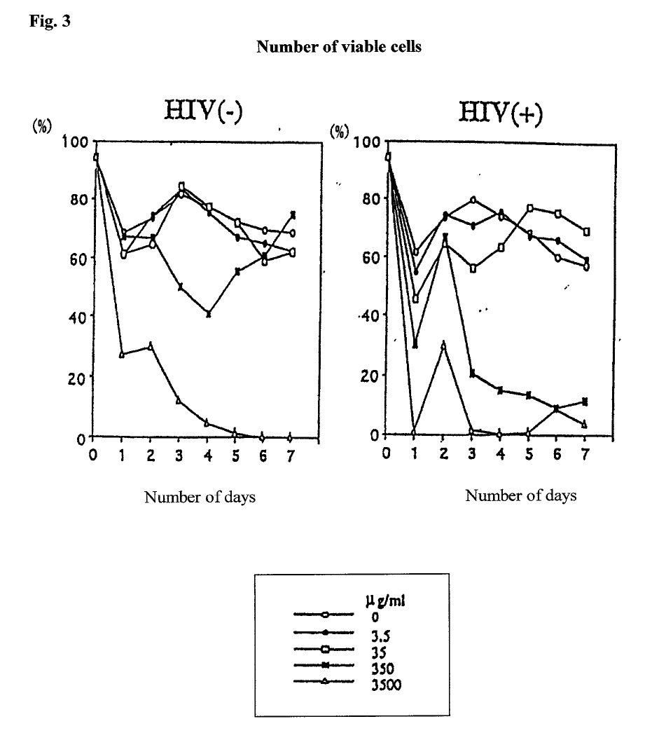

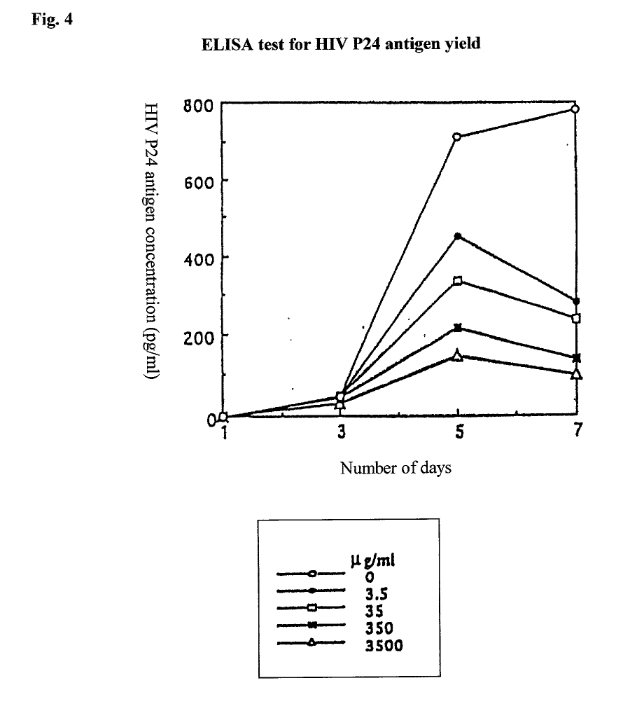

Next, it was determined how Kabanoanatake of the present invention affects the cell viability. The two graphs in FIG. 3 provide the results of the determination. The graph of HIV (−) expresses the results in the non-infectious systems and the graph of HIV (+) expresses the results in the infectious systems. The graph shows the viability (ordinate, %) and test period (abscissa). In the test that Kabanoanatake extracts was used at the concentration of 0, 3.5, 35, 350, 3500 μg/ml, the amount of living cells was very low at the concentration of 3500 μg/ml, despite whether it is in the infectious systems or in the non-infectious systems. FIG. 4 shows the HIV P24 antigen yield showed in Kabanoanatake extracts at the same concentration respectively (ordinate, yield concentration of HIV antigen P24 [pg/ml]; abscissa, elapsed number of days), though the low HIV P24 antigen yield is not due to the direct anti-HIV effects of Kabanoanatake, but due to killing the infected or non-infected cells. Turning to the graph of FIG. 3, the number of living cells showed in Kabanoanatake was found to decrease at the concentration of 350 μg/ml (black quadrangle) as compared to the control, but there was a difference between the infectious systems and the non-infectious systems. In other words, the number of living cells showed the tendency to decrease in the infectious systems with time, but to recover in the non-infectious systems after the 4^{th} day. In other words, the number of living cells showed the tendency to decrease in the infectious systems with time, but to recover in the non-infectious systems after the 4^{th} day. This suggests the possibility of Kabanoanatake extracts to specifically kill the infected cells at the concentration of 350 μg/ml and to contribute to the activity of phagocyte in the non-infectious systems. The number of living cells as well as viability showed in Kabanoanatake extracts at the concentration of 35 μg/ml, 3.5 μg/ml were almost the same as that of control, and as is shown in the graph of FIG. 4, the HIV P24 antigen yield was well suppressed. It may result from the anti-HIV effects of Kabanoanatake.

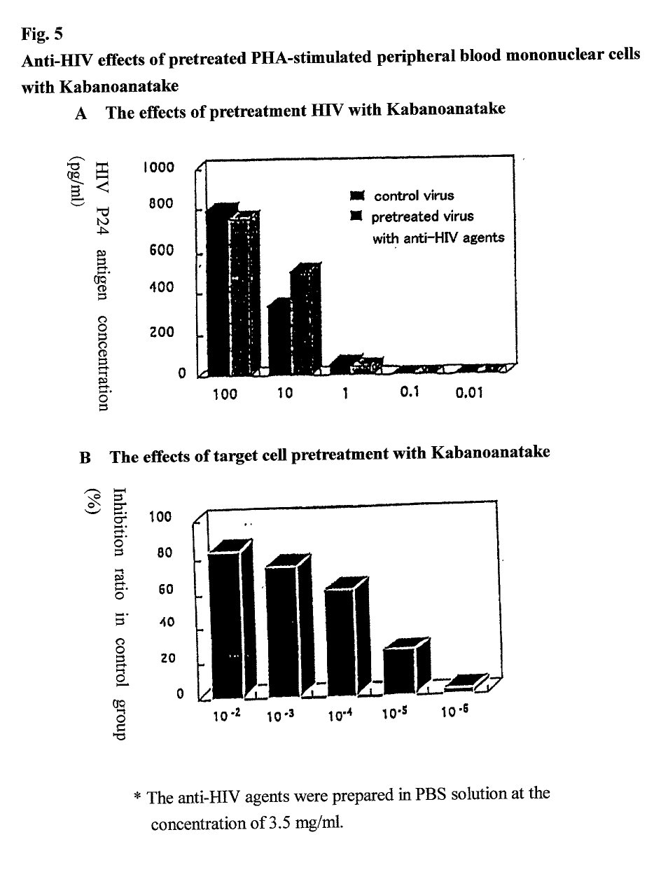

Next, in the present invention, a test was run for examining whether Kabanoanatake show anti-HIV effects or not by means of direct action against HIV. The graph A, B of FIG. 5 show the results of this test. First, Kabanoanatake and the virus were pretreated for two hours, then ultra-centrifuged at 45000 rpm for 90 minutes to remove Kabanoanatake. The virus was made infected with PHA (phytohemagglutinin)—stimulated normal human peripheral blood mononuclear cells (PBMC). As the graph A illustrates the results, it had an almost similar infectivity as the control virus (ordinate, the concentration of the HIV P24 antigen pg/ml; abscissa, the concentration). The point is that the anti-HIV effects of the Kabanoanatake were considered not to affect the virus directly. Next, pretreatment of Kabanoanatake and PHA-stimulated PBMC for two hours, were made infected. As illustrated in the graph B, the infection was inhibited depending on concentration at the concentration whereby the viability was relatively maintained in the previous test.

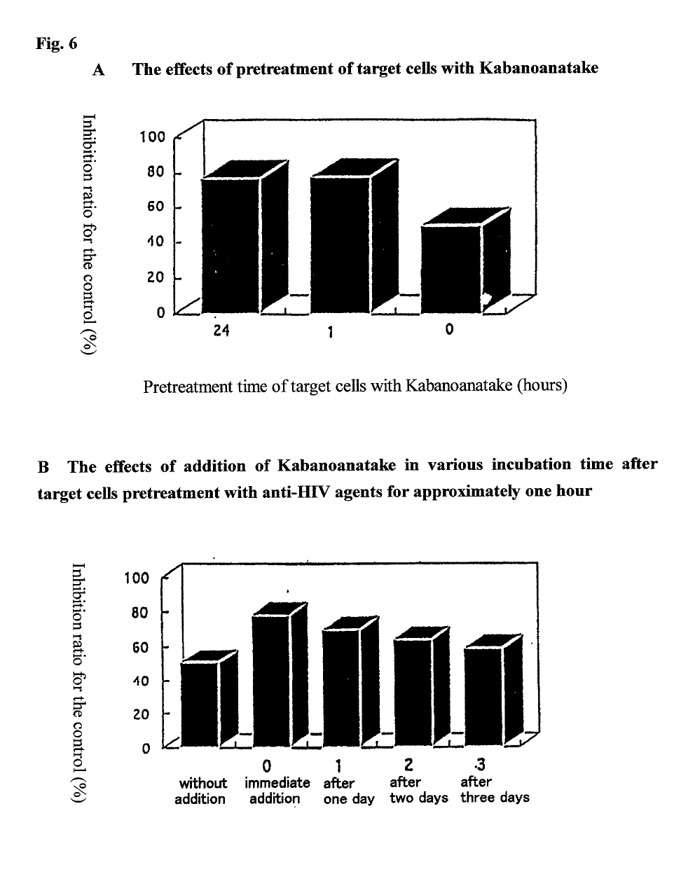

Furthermore, infection inhibition efficacy was determined regarding the cases that PHA-stimulated PBMC was pretreated with Kabanoanatake for 24 hours, one hour, and was not pretreated. The graph of FIG. 6 illustrates the results. The procedure concerning the graph A that 100TCID_{5 0} of the HIV virus was made infected in the presence of Kabanoanatake after pretreatment, washed and then incubated by a culture solution containing Kabanoanatake to measure the HIV P24 antigen released on the 5^{th} day (ordinate, inhibition ratio, %; abscissa, the pretreatment time for target cells by Kabanoanatake). As a result of that, even in the case pretreated for one hour, the activity more than 70% was observed, as is so in the case pretreated for 24 hours. Furthermore, it was elucidated that 50% of the infection inhibition was observed in the case that was not pretreated. In the graph B of FIG. 6, the infection inhibition effects of Kabanoanatake on the virus were shown, where Kabanoanatake was added to PBMC pretreated for one hour and infected with virus in the same manner, on the first, the second and the third day after infection. As a result of that, it appeared that when Kabanoanatake was added to PBMC at a later date, the infection inhibition effects decreased more or less, although even in a culture solution, which does not contain Kabanoanatake, infection was inhibited by 50%. For reasons mentioned above, the active ingredients of Kabanoanatake of the invention may be considered to exert anti-HIV effects when an infection starts. It was also clarified that the pretreatment was more capable of inhibiting infection. Thus, some action of Kabanoanatake on the side of the cells is presumed.

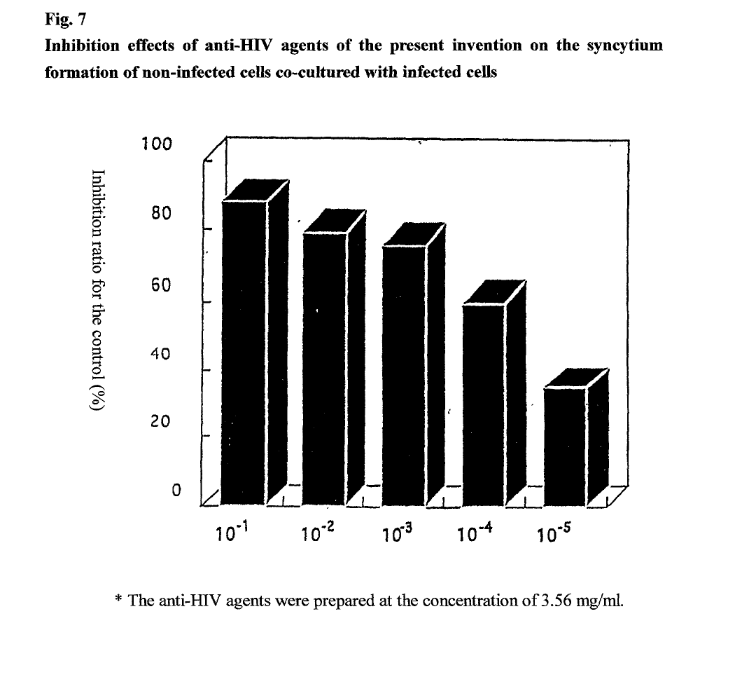

FIG. 7 shows the suppressive effect on the syncytium formation by anti-HIV agents of the present invention, when Molt 4/c 18 cells (non-infected cells) are cultured together with Molt 4/HIV IIIB cells (infected cells). Correspondently, Kabanoanatake appeared to reduce the syncytium formation depending on the concentration. Kabanoanatake was considered to deeply relate to viral entry steps in HIV.

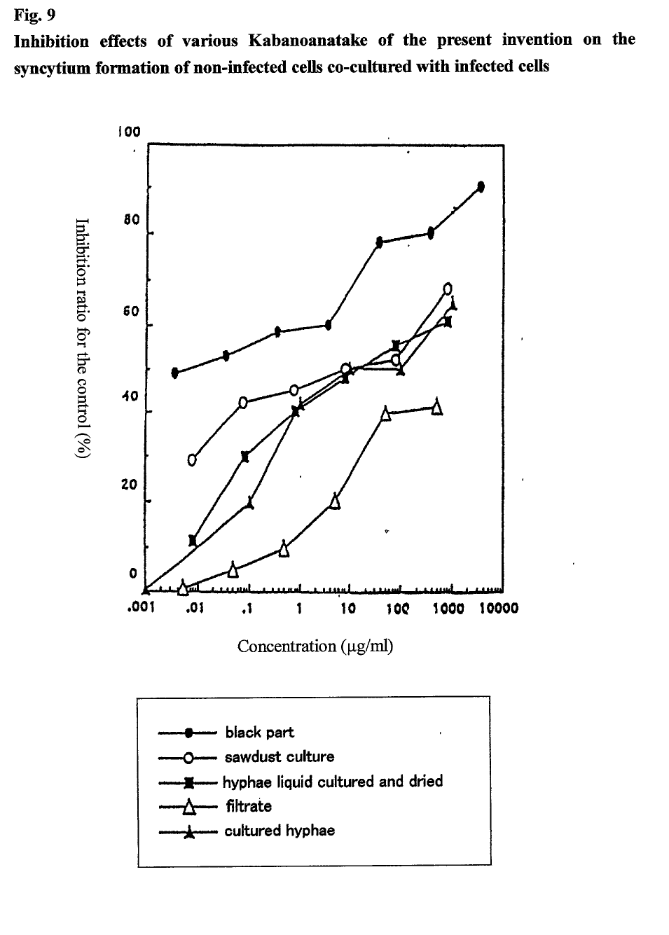

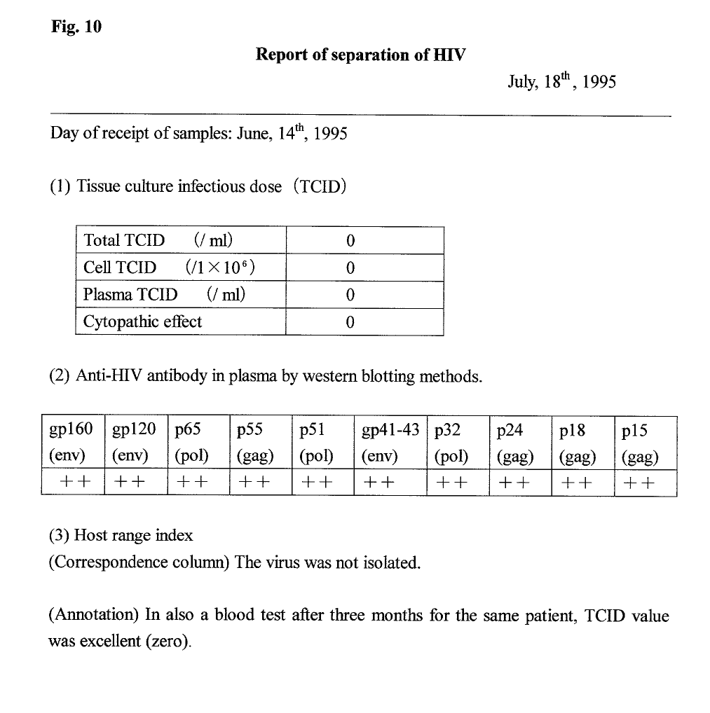

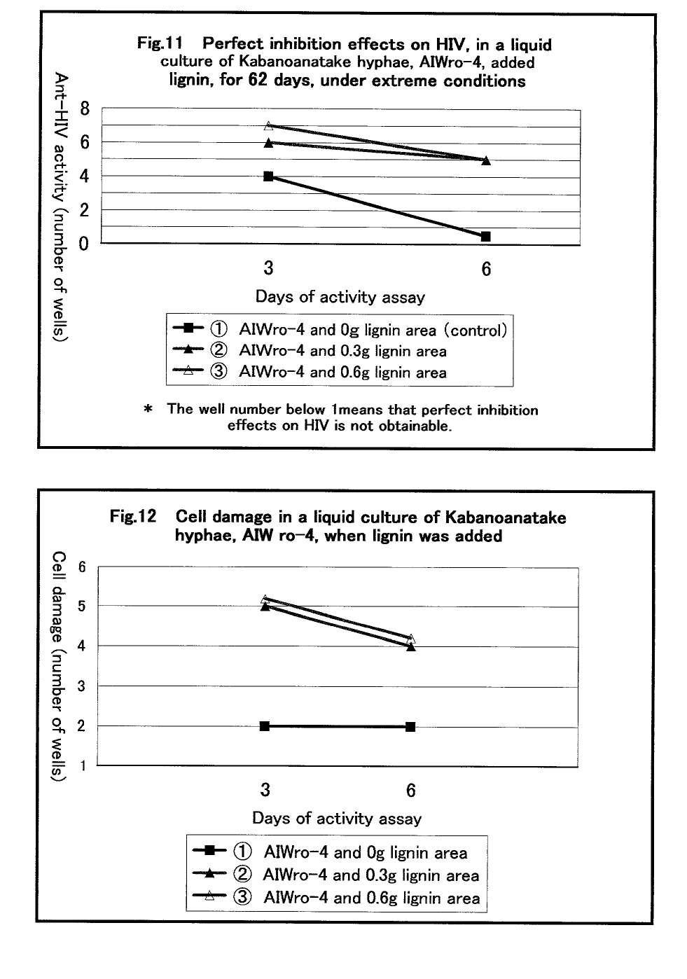

As indicated in the next FIG. 9 (suppressive effect on the syncytium formation), the present invention established the culture conditions whereby the strain similar to that of natural Kabanoanatake can be obtained, as the results of attempting to culture Kabanoanatake under various culture conditions, with the object of producing on a large scale of active factor having anti-HIV effects of Kabanoanatake. Among these, using, in particular, extracts obtained by sawdust culture (white circle) and liquid culture (black quadrangle), as well as the filtrates (white triangle), the suppressive effect on the syncytium formation were determined. The results are given in FIG. 9, along with the result in the case of black part of natural Kabanoanatake (black circle). The graph of FIG. 9 shows the results involving the suppressive effect on the syncytium formation by various anti-HIV agents of the present invention, when Molt 4/c18 cells (non-infected cells) were cultured together with Molt 4 /HIV IIIB (infected cells). The ordinate indicates the inhibition ratio (%), and the abscissa indicates the concentration of the anti-HIV agents. Consequently, of the five kinds of anti-HIV agents (the black part of Kabanoanatake, sawdust culture, hyphae cultured in liquid and dried by heating, filtrates, cultured hyphae) inhibited the syncytium formation, depending on the concentration, and ED50 being observed as to natural Kabanoanatake (black circle) in around the extremely small amount of 35 ng/ml (0.001 μg/ml), the inhibition ratio being increased dramatically with the concentration. The extracts obtained by culture appeared to inhibit 50% or more of syncytium formation, as well. Furthermore, filtrates (white triangle) appeared to have the inhibition effects against 40% of syncytium formation.

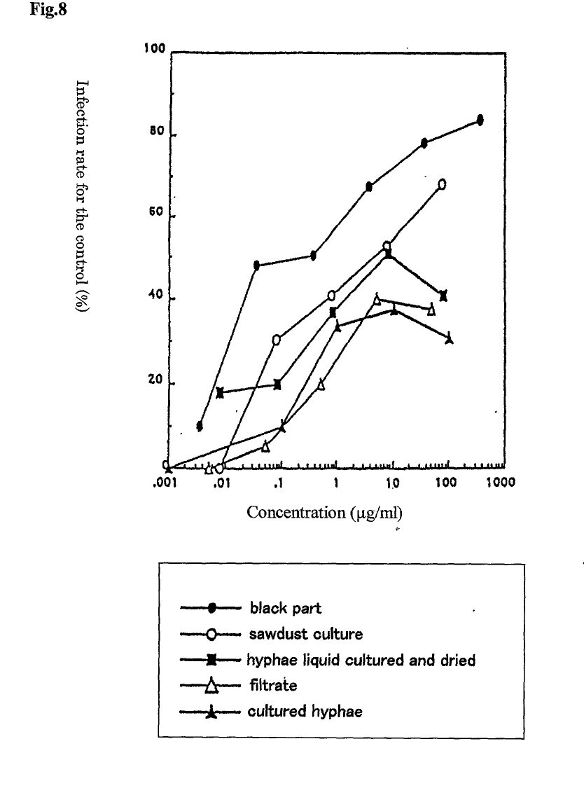

As has been discussed, sawdust-cultured Kabanoanatake and liquid-cultured Kabanoanatake in addition to natural Kabanoanatake of the present invention possess anti-HIV effects respectively. The graph of FIG. 8 contains the more detailed information on the test of infection inhibition efficacy. (The ordinate indicates the inhibition ratio (%), the abscissa indicates of the concentration of the anti-HIV agents.) The test of infection inhibition efficacy regarding five kinds of anti-HIV agents was performed at various concentrations as is shown in FIG. 8, by means of the samples used in the said Fusion assay. As a result of that, it was recognized that there exist inhibition activity against infection in correlation with inhibition activity against the syncytium formation. Even in the case of the extracts obtained by culture, inhibition activity against infection was observed to a considerable extent, although it is lower than that of natural Kabanoanatake (black circle). In particular, the black part of Kabanoanatake showed the inhibition ratio of 50% at low concentrations which was not expected previously, for instance, even at the concentration of approximately 0.01 μg/ml. It revealed more potent activity in far lower concentrations than the activity concentration shown in Lentinus edodes, Ganoderma lucidum, Flammulina velutipes, Auricularia, etc. described above in relation to the published examples.

It was recognized that the active ingredients extracted from Kabanoanatake which concerned with the present invention are extremely effective anti-HIV agents, and practically the efficacy thereof are able to be enhanced when the active ingredients are used in combination with the medicinal properties of Chinese herbal medicines. For example, the medicinal properties of Chinese herbal medicines such as Lithospermum erythrorhizon (roots), Cinnamomum sieboldi, Prunus persica, Pinellia ternate, Poria cocos, Aconitum japonicum Thunb (manufactured goods), Daucus carota (leaves), Glycyrrhiza uralensis, Schisandra chinensis, Zingiber officinalis, Asarum sieboldii, Prunus armeniaca and Rheum palmatum, etc. used as a mixture or concomitantly with some other agents may be combined or coupled with the active ingredients of Kabanoanatake of the present invention, and in fact the combined use of Lithospermum erythrorhizon (roots), Cinnamomum sieboldi, Prunus persica with the active ingredients extracted from Kabanoanatake particularly have been shown to be effective in HIV inhibition when used in combined. It is known that other bacillary virus infection of human cells activates the latent HIV virus, which acts as a trigger for HIV to start to grow and develop symptoms. In order to suppress the activation of HIV occurred by such mixed infection, in particular, the combined or concomitant application of one ore more ingredients of Chinese herbal medicines including Lithospermum erythrorhizon, Cinnamomum sieboldi, Prunus persica, etc. with the active ingredients of Kabanoanatake may prevent the internal organs such as liver, kidney of the persons who take them from bacillary virus, and it may also prevent HIV from developing by purifying and strengthen the organs, whereby the active ingredients of Kabanoanatake can reach its potential.

As a practical matter, the active ingredients of Kabanoanatake which are concerned with the present invention are able to be extracted by various solvents or various extraction methods. For instance, hyphae of natural Kabanoanatake or cultured Kabanoanatake were treated with PBS solution, butanol, ethyl acetate or acetone to give the active ingredients from each insoluble matter.

The active ingredients thereof may be taken out of each non-adsorbent by adsorbing and treating the components or extracts from hyphae of natural Kabanoanatake or cultured Kabanoanatake by carbon or charcoal. Besides, the active ingredients may be extracted by even boiling natural Kabanoanatake or cultured Kabanoanatake in hot water at various pH levels (e.g. for 60 minutes). The extracted active ingredients obtained by the present invention were recognized to be stable substances which are soluble in water, thermoduric and acid resistant and in any case to show inhibition ability against the syncytium formation by HIV and infection inhibition efficacy at an almost equal level. Kabanoanatake which is used in the present invention include, in addition to the said sawdust-cultured or liquid-cultured Kabanoanatake, hyphae and its secretions proliferated artificially by planting Kabanoanatake hyphae on the green wood of birch trees, or what was made to form sclerotia artificially by planting Kabanoanatake hyphae on the green wood of birch trees. These Kabanoanatake produced by artificial planting of hyphae are more of the ones which are near to natural products than cultures, accordingly included in natural Kabanoanatake in the present invention.

The effects on immunity improvement in a HIV patient who has taken active ingredients of Kabanoanatake of the present invention orally are described. After the active ingredients of Kabanoanatake of the present invention were given by mouth to the HIV-positive patient, in an early stage (60 days after administration), in the middle stage (150 days after administration), immunity code was measured by L.F.T. (Life Field Tester) in the later stage (547 days after administration). The measurement was done by analyzing the patient's first urine of the morning, using L.F.T. The immunity code was −7 on the 60^{th} day, approximately 0 on the 150^{th} day, and the improved figure, +7 was obtained on the 547^{th} day. The figure −7 is said to be at the same level as lower figures of normal persons in magnetic field analysis. In addition, the mean immunity codes in normal persons are around +13. The active ingredients of Kabanoanatake of the present invention was also proved to be effective by blood examination, as is shown in the figure, from where it is recognized that they may be effective preventive agents as well as therapeutic agents.

(II) Embodiment as Anti-Bacterial Agents

In the present invention, the anti-bacterial activity of Kabanoanatake extracts were tested and confirmed regarding pathogenic bacteria, in particular, the various bacteria listed below in Table 1, that is to say, Escherichia coli O157,Klebsiella pneumoniae, Pseudomonas aeruginosa, Staphylococcus aureus, Bacillus subtilis, Clostridium perfringens, according to minimum inhibitory concentration method (MIC) (Japanese Society of Chemotherapy). Aside from Clostridium perfringens, each seed fungi of specified amount was inoculated into a medium for sensitivity disks (the numbers of inoculated bacteria, Table 1), and Clostridium perfringens was tested by inoculated into Brain-Heart agar culture medium added 0.1% sodium thioglycollate, using the method described.

One way to produce Kabanoanatake extracts using anti-bacterial activity test in the present invention is as follows. In the first place, 100 g of powdered Kabanoanatake (natural or artificial cultures), for instance, is placed in 1000 cc of distilled water, and extracted by hot water at the temperature of 100° C. for 40 minutes. In the second place, the extract was filtrated through gauze or paper filter, and was centrifuged. In the second place, the extract was filtrated through gauze or paper filter, and was centrifuged. It was centrifuged, for example, by a separator at 3000 rpm for 10 minutes. Furthermore, it was filtrated again and freeze-dried to obtain the active ingredients of Kabanoanatake extract aimed at. The effects of this anti-bacterial activity test are given in Table 1.

TABLE 1

| | Numbers of | |
|---|---|---|
| Pathogenic bacteria | inoculated strains | MIC (ppm) |
| MRSA (*) | | 5,000 |
| (methicillin resistant | | |
| Staphlococcus aureus) | | |
| E. coli O-157 (EDL 931) | 2.7 × 10⁷ | 10,000 |
| K. pneumonise (ATCC 4352) | 8.0 × 10⁶ | 10,000 |
| P. aeruginosa (IFO 13276) | 2.8 × 10⁶ | 5,000 |
| S. aureus (IFO 12732) | 2.2 × 10⁷ | 5,000 |
| B. subtilis spore (ATCC 6633) | 1.4 × 10⁶ | 100,000 |
| C. perfringes H2 | 2.0 × 10⁶ | 2,600 |

(*) After cultured on SOD medium at 37° C. for a night, methicillia resistant Staphlococcus aureus was prepared to 10⁶ CFV/ml, incubated into Mueller-Hinton agar medium containing test specimen (Kabanoanatake extract) in a two fold dilution series, using Micro-Planter, and cultured at 37° C. for 18 hours, to give minimum inhibitory concentration.

To ascertain as to whether the anti-bacterial activity is a peculiar property to Kabanoanatake or other mushrooms have similar anti-bacterial action, the following test was performed. Candidate mushrooms included Granolas frondosa (Dicks:Fr), Lentinus edodes (Berk) Sing. in addition to Kabanoanatake. 100 g of raw materials of these candidate mushrooms were placed in a vessel, respectively, with 1000 cc of distilled water, and were boiled at 100° C. for 40 minutes to obtain extracts. By filtrating these extracts through Toyo filter paper No. A5, 200 cc extracted test liquid was obtained from each candidate mushroom. Anti-bacterial test was run as regards these extractives, by means of medium for sensitivity disks, through the method, similar to the one described above. The results are shown in Table 2.

TABLE 2

| | | Numbers of | |
|---|---|---|---|
| Samples | Types of bacteria | incubated stains | MIC |
| (1) | E. coli O157 (EDL 931) | 2.7 × 10⁷ | 10,000 ppm |
| (2) | E. coli O157 (EDL 931) | 1.0 × 10⁶ | not hampered |
| (3) | E. coli O157 (EDL 931) | 1.0 × 10⁶ | not hampered |

Sample (1) = Kabanoanatake extracts boiled by hot water
Sample (2) = Grifola frondosa
Sample (3) = Lentinus edodes
Sample (3) = Lentinus edodes

Anti-bacterial activity tests of Kabanoanatke cultures were performed concerning helicobacter pylori. The results appeared to be effective. The procedures are as follows.

1. Samples

As samples, three kinds of natural Kabanoanatke (extracted with hot water), cultured Kabanoanatke (extracted with hot water) and lignosulfonic acid sodium salt were used.

2. Adjustment of Samples

Each sample was extracted using DMSO at 10 w/v % to make stock solution, further from which ten-fold serial dilution was made by DMSO, to prepare the test solution.

3. Helicobacter pylori Culture and Adjustment of Strain Suspension

H. pylori. strain NCTC 11637 and strain NCTC 11916 were cultured on blood agar which added 5% defibrinated horse blood to Brucella Agar (BBL) at 37° C. for 3 days, under microaerophile surroundings (N₂80%, C0₂15%,0₂5%). The strains were suspended in Brucella broth added 0.5 mg/ml of BSA fraction V (Sigma A-4503) to a concentration of 10⁸ cfu/ml, to prepare inoculum organism liquid, and 0.1 ml of the liquid was smeared on BSA agar added the said BSA, after that anti-bacterial activity against H. pylori was determined by Disc method, as described below.

4. Disc Diffusion Method

Each test solution of 20 μl was made to absorb in a sensitivity disk (8 mm in diameter, Advantech, thin-model), placed on agar smeared with strains, cultured under microaerophile surroundings at 37° C. for 3 to 5 days. After that, growth-inhibitory zone around the disk showing more than 1 mm was regarded as positive.

5. Results

The results are as follows.

TABLE 3
The results of a test involving antibacterial
activity of each sample, using extracted
DMSO stock solution. Antibacterial activity: +, presence; −, absence

H. pylori strain number

| Samples | 11637 | 11916 |
|---|---|---|
| 1 Cultured Kabanoanatake | + | + |
| 2 Natural Kabanoanatake | − | − |
| 3 Lignosulfonic acid sodium salt | − | − |

| | MIC (μg/ml) | | | |
|---|---|---|---|---|
| Control (reference) | 11637 | | 11916 | |
| antibiotics test method | blood agar | BSA agar | blood agar | BSA agar |
| Erythromycin Disk* | 6.3 | 3.1 | 6.3 | 1.6 |
| agar plate dilution | 0.05 | 0.03 | 0.05 | 0.03 |
| method | | | | |

*Growth-inhibitory zone around a disk showing more than 1 mm is regarded as minimum concentration (Minimum Inhibitory Concentration)

Although the basis of evidence that Kabanoanatake have anti-bacterial properties, which are not seen in other mushrooms, has not been grasped definitely, especially regarding the biological activity for keeping human bodies from bacteria known as immunostimulating effects. Cells concerning immunology may be lymph corpuscle and macrophage, LPS (lipopolysaccharide) contributing to the macrophage activity. The test was conducted in order to ascertain how much of the constitution of the Kabanoanatake is recognized to be important by the present inventor, the results are given in the Table 3. As samples, carrot extract and carrot leaf extract were used in addition to Kabanoanatake. To prepare the samples, separate portions of 500 g of Kabanoanatake, carrot extract and carrot leaf extract were weighed and distilled water for injection were added to them to a final concentration of 100 mg/ml. These were extracted by heating at 56° C. for three hours in hot water, and then centrifuged, of which the supernatant was taken to make into the stock solution for measuring samples. LPS was determined by colorimetry, Limulus amoebocytelysate assay (Seikagaku Corporation) to measure LPS specifically.

TABLE 4

| Samples | LPS content (μg/g) |
|---|---|
| Kabanoanatake extract | 1.601 |
| Carrot extract | 0.85 |
| Carrot leaf extract | 29.5 |

(III) Embodiment of Liquid Culture Method

(1) Practical Incubation

According to the principals of the present invention, the details of the practical liquid culture method are described hereafter. Although incubation vessels are not limited to the specified ones, 5 L erlenmeyer flask is suitable for convenience in handling and ease of manageability. The following is the example of the standard medium culture composition (liquid).

malt extract (Baltimore Biological Laboratory) 20 g

glucose 20 g

peptone (Nihon Seiyaku) 6 g

yeast extract (Oriental Yeast, Co., Ltd.) 6 g

1/100M potassium phosphate buffer (p H5.0) 200 ml

water (tap water, ground water or snow water) 1800 ml

Thus, 2 liters of standard liquid medium can be made. They all are placed into a 5 L erlenmeyer flask to make a liquid medium. When incubation vessels are larger or smaller than this, a liquid medium may be prepared in proportion to the said combination ratio. The flask containing liquid medium is closed with the silicon stopper loosely placed on the flask. Part of the silicon stopper is covered with aluminum foil. The medium flask should be autoclaved at 121° C. for 20 minutes for sterilization heating. After the flask is cooled, the silicon stopper is sealed tightly, which had better be done on a clean bench, to make doubly sure. The large incubation volume may be using many medium flasks at the same time. The said Kabanonatake hyphae incubated before test is added to these medium flasks, and then incubated. Actually various strains are used, but hereunder in Table 5, the several values regarding selected three typical bacterial strains, A-2, A-6, A-7 are shown. Furthermore, in these cases, the fluid volume at the initiation of incubation was 200 ml in a 500-ml Sakaguchi flask.

TABLE 5

| Kabanoanatake | Culture | pH | Protein | Glucose | Brix | | Final liquid measure |
|---|---|---|---|---|---|---|---|
| strains | days | (liquid) | mg/ml | mg/ml | % | 500 nm | (114^{th} day) |
| A-2 | 28 | 7.26 | 0.135 | 0.093 | 0.8 | 1.4323 | 140 ml (70%) |
| | 70 | 8.46 | 0.2338 | | 1.2 | 1.390 | |
| | 114 | 8.04 | 0.416 | 0.162 | 1.1 | 1.8802 | |
| A-6 | 28 | 5.41 | 0.058 | 0.093 | 1.3 | 0.5187 | 140 ml (70%) |
| | 70 | 8.15 | 0.2610 | | 1.0 | 1.694 | |
| | 114 | 7.92 | 0.404 | 0.058 | 1.0 | 1.8592 | |
| A-7 | 28 | 6.22 | 0.227 | 0.140 | 1.3 | 2.1426 | 142 ml (72%) |
| | 70 | 8.15 | 0.4093 | | 1.0 | 1.751 | |
| | 114 | 8.04 | 0.494 | 0.100 | 1.1 | 1.9272 | |

Anti-HIV virus activity was measured by the test method using the said 96 well microplate regarding these three kinds of bacterial strains on the 28^{th}, 70^{th}, 114^{th} day. The effects are described below along with other findings. 100% inhibition activity against HIV was examined in a two-fold dilution series.

A-2:500 nm absorbance was 1.4323 on the 28^{th} day of culture, which was a good beginning. It decreased somewhat to 1.390 on the 70^{th} day, but increased to 1.8802 on the 114^{th} day of culture. In the meantime, the effective dosage for anti-HIV activity was 250 (on the 28^{th} day), 250 (on the 70^{th} day) and 62.5 μl/ml (on the 114ₜₕ day).

A-6: Due to its low colored secretions, 500 nm absorbance was 0.5187 on the 28^{th} day, which was not a good beginning. 500 nm absorbance increased to 1.694 on the 70^{th} day, and increased further to 1.8592 on the 14^{th} day. The effective dosages for anti-HIV activity during the periods were zero (on the 28^{th} day), 125 (on the 70^{th} day) and 62.5 μl/ml (on the 114^{th} day).

A-7: 500 nm absorbance increased dramatically to 2.1426 on the 28^{th} day of culture. Afterwards, it was lowered to 1.751 on the 70^{th} day, which increased again to 1.9272 on the 114^{th} day. The effective dosages for anti-HIV activity were 125, 31.25 and 125 μl/ml.

On the whole, in the liquid culture method of the present invention, black color showing the active ingredients appears on approximately between the 25^{th} to the 33^{rd} day. This indicates that the active ingredients may be obtained in a relative short period of time. However, there are some incubated areas where the production of the active ingredients are low, which is improvable by adopting measures such as interfusion of lignin substances as wood constituents (described later according to the present invention) into a culture medium, addition of humic acid, or increase in the amount of culture medium.

Analysis Method

As common practice, the following method has been adopted. Each sample was counted to an accuracy of 200 mg/ml, and dissolved in deionized water. Stirred thoroughly and centrifuged at 3,000 rpm for 15 minutes, the supernatant solutions thereof were taken and subjected to the following test.

Experiment Equipments

spectrophotometer; Shimazu spectrophotometer UV-1200

p H meter; Toua Digital p H meter-50

A. OD 500 nm and p H

After the preparation of 1% (w/v) aqueous solutions of each sample, the absorbance was read at 500 nm and p H was measured, using a routine method.

B. Protein

The protein content in each sample solution was assayed by the Bradford method. The sample solution, 20 μl, was placed into a 1.5 ml test tube and was combined with 1 ml of Bradford solution. The mixture was left standing at room temperature for 5 minutes, and then the absorbance was read at 595 nm. As a control group, Bradford solution added 20 μl deionized water was employed. The sample solution was diluted ad libitum and measured, being careful not to be affected with impurities contained in the samples. The protein content in each sample solution was calculated, referring to a calibration curve created using bovine serum albumin as a reference, and converted into the amount per 1 g sample.

C. Glucose

Using glucose C-II Test Wako (Wako Pure Chemical Industries, Ltd. ), the glucose content in each sample solution was measured. The reagent used for this measurement was an enzymatic reagent with high specificity. The sample solution, 20 μl, was placed into a test tube and was combined with 3.0 ml color-producing reagent. After warmed to 37° C. for 5 minutes, the absorbance of each solution was read at 505 nm. As a control group, sample solution added 3.0 ml of deionized water was employed, thus the color effect was eliminated. Calibration curves were produced by simultaneous reaction to the glucose standard solution. The glucose content in each sample solution was calculated, referring to the calibration curve and converted into the amount per 1 g sample

D. All Saccharides

Using a phenol-sulfuric acid method, the total amount of saccharide in each sample was measured. The sample solution, 20 μl was placed into a test tube and was combined with 5% phenol solution, 200 μl. Then, 1 ml of concentrated sulfuric acid was added dropwise to the test tube, and stirred rapidly. After left standing at room temperature for 20 minutes, the absorbance was read at 490 nm. As a control group, distilled water was used instead of the sample solutions. Calibration curves were produced by simultaneous reaction to the glucose standard solution. The sample solution was calculated as the amount of glucose, referring to the calibration curve and converted into the amount per 1 g sample.

(2) Addition of Sap of White Birch

According to the said liquid culture composition, the effects on the addition of sap of white birch to water was determined. The sap of the white birch was added by 5%, 10%, 40% and 90% in total. They were incubated for 42 days along with the control, to which the said saps were not added, and observed about Brix (%), the amount of dried hyphae (g), the quantity of glucose and protein (mg/ml), redox potential (mV), and pH, as indexes. As a result, at the concentrations of more than 5%, the increasing amount of dried hyphae of Kabanoanatake was seen. Redox potential was also low at the concentration of more than 40%, and the amount of protein increased. Through these results, it was found that the sap of the white birch has a good effect on the liquid culture, by adding at least 40%.

(3) Incubation by Snow Water

Moisture is essential to the culture solution. To ascertain that the culture results depend on the different quality of water, water from melted snow was used to culture Kabanoanatake. The snow of the Nayoro region in Hokkaido where the inventor is domiciled is of much better quality, compared to that of other regions. Another study of the present inventor proved that it helps the plants such as tulips grow better, and improves the skin texture of human beings. We sell this snow water sold under the trademark of “Yukinohada (skin of snow)”. The incubation of hyphae of Kabanoanatake was conducted by means of this snow water (100%) and ground water of the said Nayoro region. The used strains for the both are the same. The culture composition is essentially equal to that of the said (3), except for using snow water. The effective dose for perfect inhibition of HIV during specific time period in which CD₄ cells were co-cultured with the HIV virus was investigated in a two-fold dilution series. It was determined to be more than 62.5 μl/ml for the samples cultured in snow water and to be more than 500 μl/ml for the controls (ground water) on the 3^{rd} day. The former was determined to be more than 125 μl/ml, and the latter more than 500 μl/ml on the 7^{th} day. From this fact, secretions from the hyphae cultured in snow water was seen to have as much as four times the activity of that cultured in ground water. With respect to cell damage of both samples, the former was confirmed to be 250 μl/ml, the latter to be 1000 μl/ml, whereby the safety of cultured Kabanoanatake is assured.

(4) Addition of Wood Constituents (Lignin Substances, Betulinic Acid, etc.)

In the said liquid medium method of Kabanoanatake, in particular, fluctuation of the incubation room temperature under the influence of outdoor air temperature in winter (33° C. to 8° C.), etc. may cause growth delay in some hyphae, which may be a causative of melting and killing hyphae as the days pass. If it occurs prior to the secretion of the active ingredients, it may be troublesome in cultures. The inventor has also researched in earnest, as the subject matter, the way to initiate anti-HIV activity in ideal incubation conditions more stably in a short period of time. Thorough the investigation, addition of lignin substances, etc. contained in wood constituents as the major constituents into the said liquid medium revealed to energize hyphae of Kabanoanatake, without reducing its vigor, and to help anti-HIV effects thereof to be more stable.

In regards to wood constituents, there are lignin sulfonic acid, lignosulfonic acid sodium salt, lignosulfonic acid sodium salt acetate, lignin alkali, lignin organosolv, lignin organosolv acetate, 2-hydroxypropyl ether, lignin hydrolytic, hydroxymethyl derivative, lignin organosolv propionate, betulin (betulinic acid), or lignin salts, etc. In such lignin substances, lignin salts are included. It is advisable that, in liquid cultures, the property of the substances be soluble in water as much as possible, although it is not limited to this property. Betulin becomes soluble when cultured with Kabanoanatake in the culture medium. Among them, lignin sulfonic acid (Kanto Kagaku Corporation) exerted a high degree of effectiveness. In general, the wood constituents are used in the range of concentration between 0.00001% and 0.00075% (weight) for the said liquid medium. For instance, in the case where lignin substances were used at 0.000293% (weight percent) by preference, anti-HIV effects could be yielded in the initial stage in a short time and high activity had been kept after the days passed. However, in the case where the weight percent was above 0.00075%, considerable growth suppression was seen in the hyphae on the liquid medium. Hereafter, three types of Kabanoanatake hyphae are explained, called under the names such as AIWro-4, A-2W-3, AIW-27, in order to distinguish them for convenience.

(a) In order to determine the perfect anti-HIV effects (100%), a neutralization assay was performed by using Kabanoanatake hyphae (AIW ro-4 hyphae incubated in the liquid medium for 62 hours), co-cultured the said CD₄ cells and the HIV virus. The experiment was conducted to examine whether the HIV virus was inhibited or not in a two-fold dilution series, by incubating Kabanoanatake at 33° C. in the daytime and 8° C. at night of room temperature and obtaining extracts thereof from the culture vessels. (Since the desired temperature for the growth of Kabanoanatake is approximately 25° C., lowering the incubation temperature is regarded as a extreme condition). In the untreated control test area, lignin substance was not added. In the control area, perfect inhibition of the HIV virus was seen at 125 μl/ml, when the concentration level of HIV was 10 TCID_{5 0} on the 3^{rd} day of the neutralization assay when HIV does not increase so much. However, on the 6^{th} day when viral amounts increased, the perfect HIV inhibition was not observed in the area containing 100 TCID_{5 0} of virus. On the contrary, in the test area added 0.3 g of a lignin substance, lignin sulfonic acid of wood constituents to the said liquid medium, which came to a total weight of 2052 g including 2 liters of water (weight percent, 0.000146%), HIV was inhibited perfectly at the concentration of 31.3 μl/ml (10TCID_{5 0}), and at 62.5 μl/ml (in 100 TCID_{5 0}), where stable inhibition effects and good growth of hyphae was observed. Similarly, in the test area added 0.6 g of lignin sulfonic acid to the said medium (weight percent, 0.000292%), the perfect inhibition was obserbed on the 3^{rd} day of the assay at 15.6 μl/ml (in 10 TCID_{5 0}), and on the 6^{th} day at 62.5 μl/ml (100 TCID_{5 0}). This test area added 0.6 g lignin sulfonic acid was recognized to show higher inhibition than that added 0.3 g lignin sulfonic acid, using 10TCID_{5 0} of virus, at an early stage of this neutralization assay.

(b) Likewise, A-2 W-3 hyphae of Kabanoanatake (cultured for 34 days in a liquid culture) was investigated by the neutralization assay under extreme conditions. In the test area without adding any lignin sulfonic acid, the perfect inhibition was seen at 250 μl/ml (10 TCID_{5 0}), but it was not seen when the viral level was 100 TCID_{5 0}. On the other side, in the area added 0.3 g of lignin sulfonic acid, 100% inhibition effect was observed at 250 μl/ml, even using 100 TCID_{5 0} of virus. Also in the area added 0.6 g of lignin sulfonic acid, the effects were recognized to increase, at 31.3 μl/ml (10 TCID_{5 0}), and at 125 μl/ml (100TCID_{5 0}).

(c) The results are described hereafter as regards a long-term culture test under extreme conditions restricting the infiltration of oxygen.

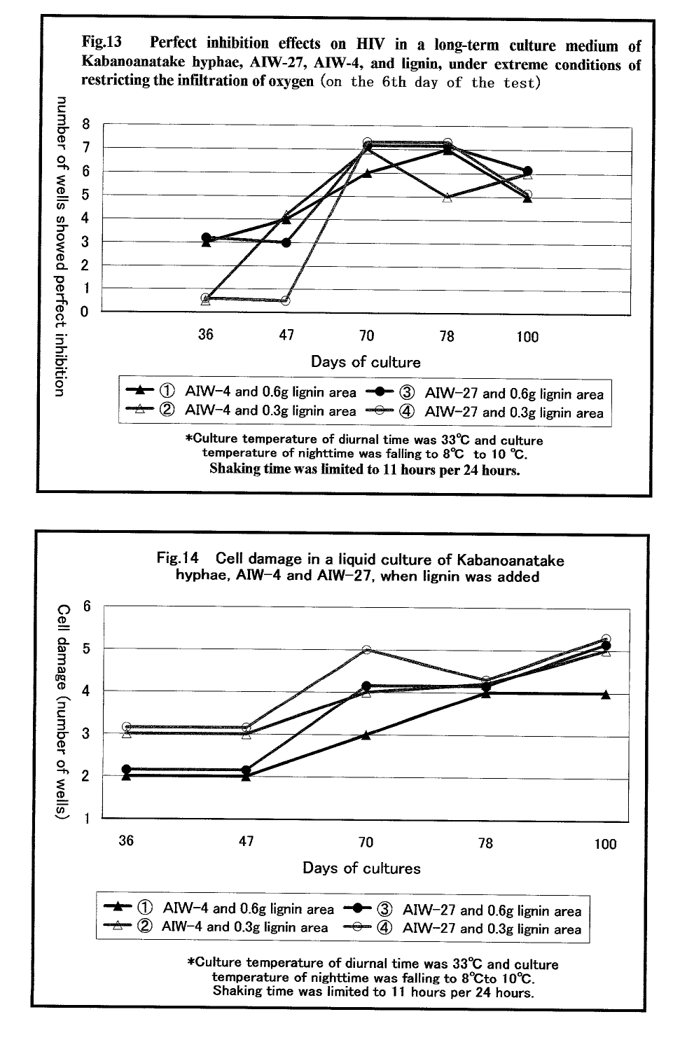

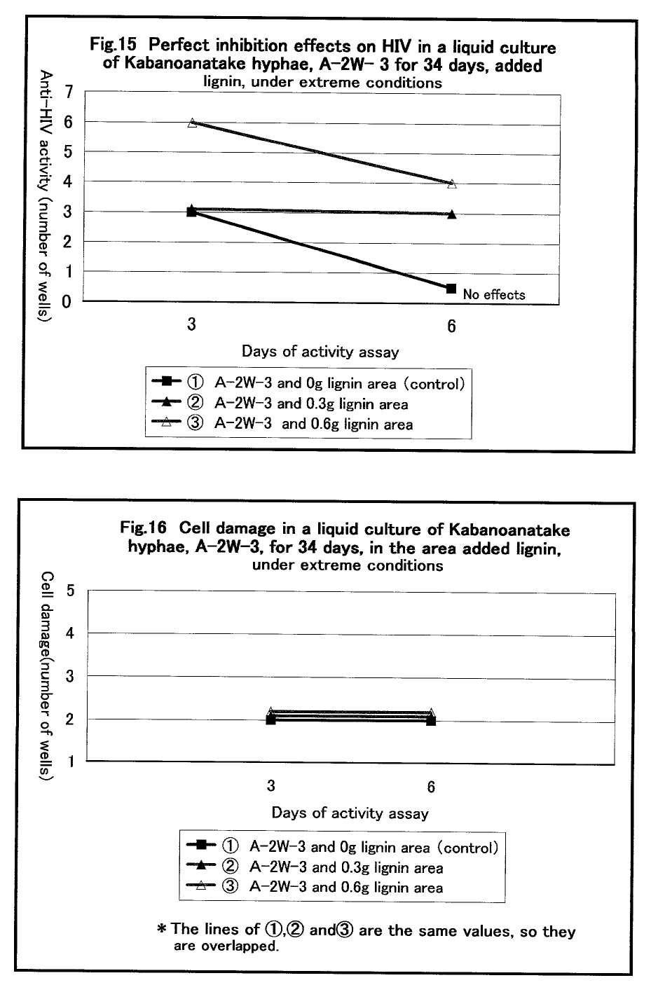

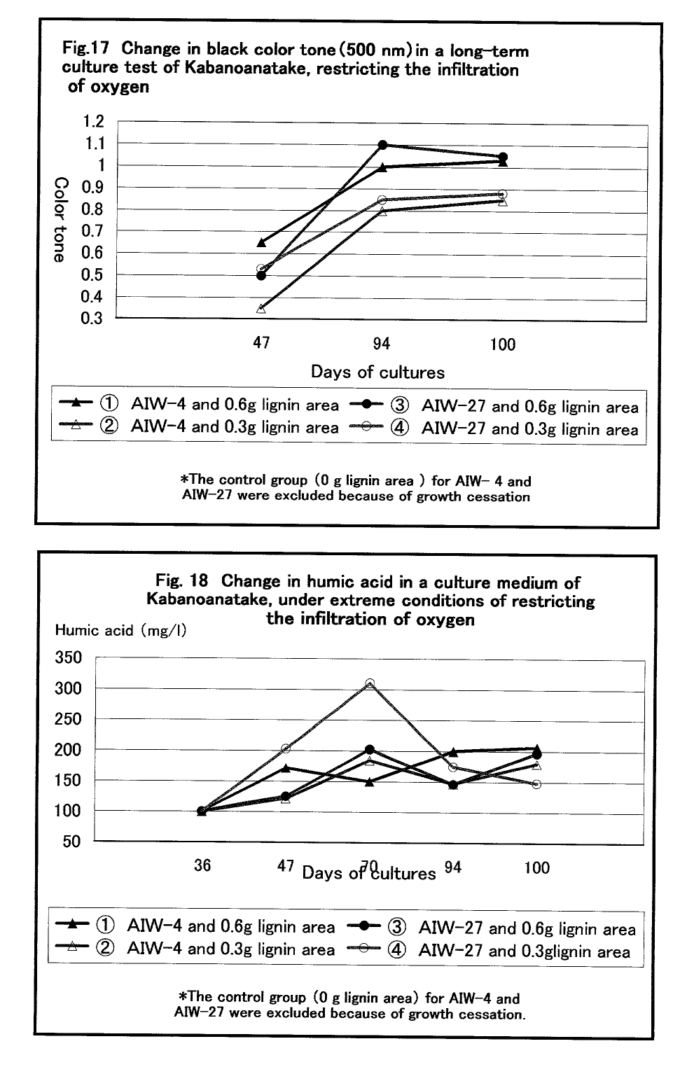

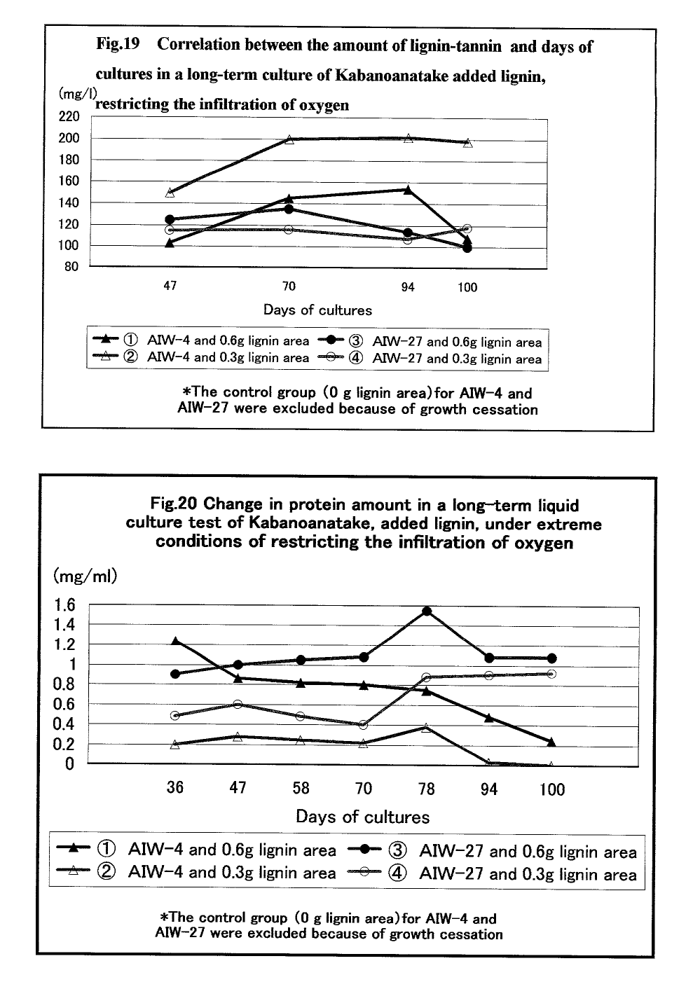

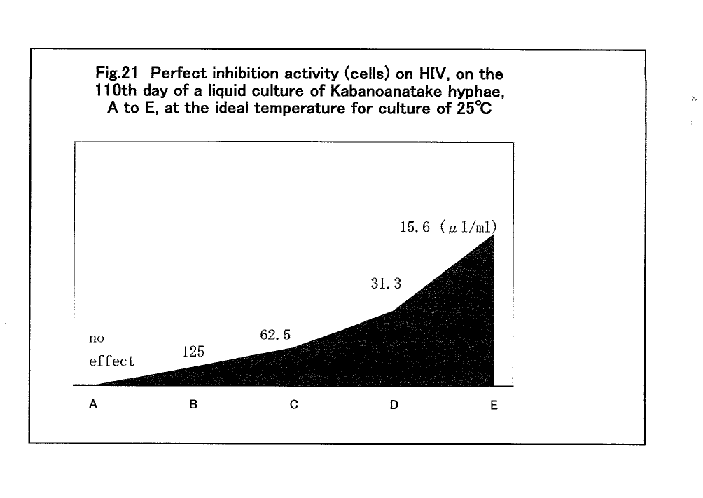

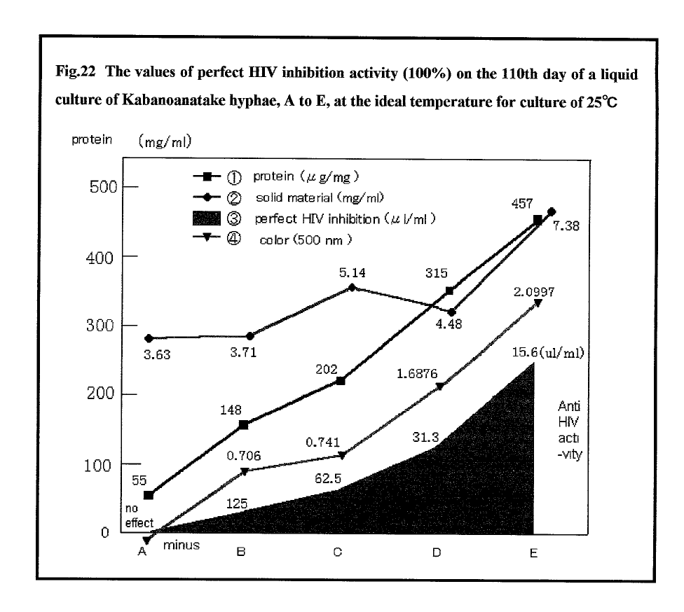

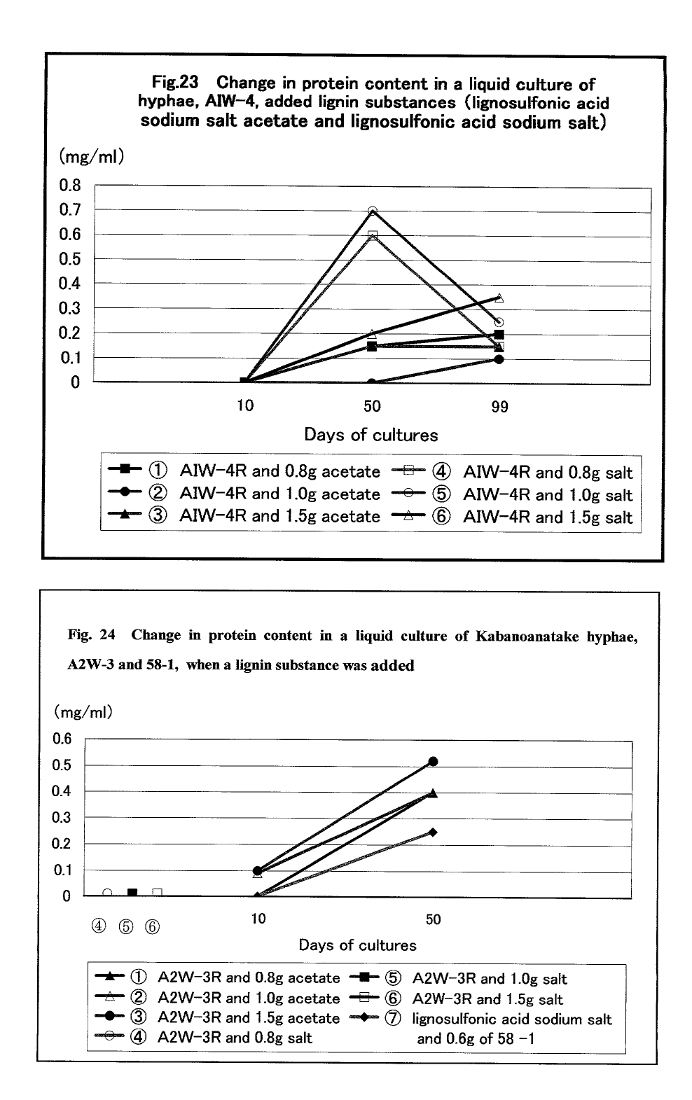

Under extreme conditions of diurnal incubation temperature of 25 to 33° C. and night incubation temperature falling to 8° C. to 10° C., the effects on liquid culture added lignin substances were investigated, using the same lignin sulfonic acid together with two kinds of Kabanoanatake hyphae (called as AIW-27 and AIW-4 for convenience), on the 36^{th}, 47^{th}, 70^{th}, 78^{th}, and 100^{th} day of culture. Practically a great number of wood constituents such as lignosulfonic acid sodium salt acetate were the subjects of investigation and proved to be effective. In the following, lignosulfonic acid is described on behalf of the wood constituents. Lignosulfonic acid is referred to as lignin hereunder. This test illustrated in FIG. 13, FIG. 14, FIG. 17, FIG. 18 and FIG. 19 was performed under extreme conditions for Kabanoanatake, of diurnal room temperature at approximately 25 to 33° C., where hyphae is incubated. 2052 g of the said culture medium and O₂ are mixed in a liter erlenmeyer flask, with reciprocal shaking at 51 cycles/min. Usually reciprocal shaking is carried out in succession for 24 hours for day, though in this investigation it was carried for only 11 hours and after that shaking was not added (extreme conditions for cultures). During winter, the incubation room is not warmed at night, so that the minimum temperature thereof becomes 8° C. to 10° C., which was utilized for this investigation. As the result of that, in the said liquid medium, two kinds of Kabanoanatake hyphae (AIW-4, AIW-27) which were not added lignin, were not able to grow under such extreme conditions, and were killed before the 36^{th} day of culture, in which the HIV inhibition was not seen. On the other hand, hyphae in all the test areas survived. Regarding anti-HIV activity, on the 36^{th} day, the perfect anti-HIV inhibition was attained at 250 μl/ml, in the test area added AIW-4 and 0.6 g of lignin as well as in the area added AIW-27 and 0.6 g of lignin, whereas it was not observed in the both area added 0.3 g of lignin. On the 47^{th} day, in the area added AIW-4 and 0.3 g of lignin, the area added AIW-4 and 0.6 g of lignin, the virus was inhibited at 125 μl/ml. In the area added AIW-27 and 0.6 g of lignin, the perfect inhibition was observed at 250 μl/ml. In the area added AIW-27 and 0.3 g of lignin, it was not seen. On the 70^{th} day when the activity dramatically increased, the HIV virus was perfectly inhibited in the area added AIW-27 and 0.6 g lignin and in the area added AIW-4 and 0.3 g lignin, at 15.6 μl/ml, and in the area added AIW-4 and 0.6 g lignin, at 32.3 μl/ml. In the area added AIW-27 and 0.3 g lignin, very high inhibition of the virus was observed at 15.6 μl/ml. Successively, on the 78^{th} day of culture, excellent inhibition activity was shown at 15.8 μl/ml in the area added AIW-27and 0.6 g of lignin, in the area added AIW-27 and 0.3 g of lignin and in the area added AIW-4 and 0.6 g of lignin. It was also shown at 62.5 μl/ml in the area added in the area added AIW-4 and 0.3 g of lignin. Furthermore on the 100^{th} day of culture, the perfect inhibition was achieved in the area added AIW-27 and 0.3 g of lignin and in the area added AIW-4 and 0.6 g of lignin, respectively, at 62.5 μl/ml, and also it was achieved at a high level of 32.3 μl/ml in the area added AIW-27 and 0.6 g of lignin and in the area added AIW-4 and 0.3 g of lignin. The present test was run under extreme conditions. Since the culture room are not warmed, the minimum temperature thereof falls to 8° C. to 10° C. during winter at night. Even under such extreme conditions, when the wood constituents were added, astonishingly the hyphae did not die and had been kept alive in all the experimental areas for a prolonged period. As already stated, excellent inhibition effects on HIV (100%) were achieved on the 78^{th} day of culture, in the area added 0.3 g of lignin, and it was also achieved even on 100^{th} day, in the areas added 0.3 g or 0.6 g lignin. It was revealed that more potent HIV inhibition activity was achieved in the area added 0.6 g of lignin (32.3 μl/ml), than in the area added 0.3 g of lignin (62.5 μl/ml). Throughout the present invention, the average ratio of solid content of Kabanoanatake hyhpae to the liquid culture was 5.31 mg/ml. For instance, when 100CTD_{5 0}, of virus is used, the effective amount, 15.6 μl/ml, is converted into 0.828 μg/ml by solid content, which indicates of 100% inhibition of HIV. Other wood constituents have a quickening effect on Kabanoanatake. Birch lignin dissolved by boiling sawdust of white birch and filtrated to be isolated from the sawdust may be added, by calculatibg the amount of solid content of lignin in the liquid medium. Addition of lignin substances including fine ground lignin and bark of birches as wood constituents to a culture medium was effective in maintaining a stable culture of Kabanoanatake, not only under extreme conditions mentioned above, but also at the ideal temperature for cultures of around 25° C. Differing from Kabanoanatake adhered to the timbers in the forests, Kabanoanatake hyphae obtained by liquid culture appeared to have instability in the quality of survival activity (pharmacologic activity). However, it has been recognized that the addition of lignins and betulin (betulinic acid) as wood constituents enables anti-HIV effects to be obtained stably, in addition it strengthens the life force of Kabanoanatake and increases its potential bioactive effects to animals and human beings, such as antimicrobial action superoxide dismutase activity and anti-mutagenicity. And this technique for utilizing Kabanoanatake is recognized as being useful, in decomposing lignin substances, according to need.

(5) The Effect of Light on Cultures

The growth level of two types of strains which are dissimilar in character, K-AIW and K-BIW were checked both in cases where they are exposed to the light or not, regarding pH, glucose content, protein content, 500 nm absorbency (production of secretion of coloring constituents), etc. Summarizing the results shortly, Kabanoanatake hyphae was proven to have a different sensitivity to light (availability) depending on the strains. For example, it is better to expose the light on K-AIW, where original protein and burnt umber components were well produced, whereas it is rather better not to expose the light on K-BIW, where more protein and coloring constituents were developed. Consequently, in ensuring the anti-HIV activity of cultured Kabanoanatake, it is important to consider the involvement of light depending on the growth level of cultured hyphae (whether exposed to the light or not), in addition to the index in judging such as coloration level in cultures, quantity of protein in the liquid medium, decrement of carbon source in the liquid medium, pH of the liquid medium, and so on. The condition for culture that allows to rapidly absorb carbon source, including glucose and other medium nutrients, thereby induce growth of hyphae by aphotic culture in initial stage of growth and then exposes to the light is confirmed to increase protein content and have higher activity in some strains (ex. K-AIW). In the above test, natural light was used as light (it had not been exposed at night), but, not applied only to this, artificial light may be used.

(6) Addition of Humic Acid

In cultures by standard liquid medium, in practice, anti-HIV activity may sometimes be lower. In order to improve the quality and yield of the cultures, a variety of studies have been conducted, wherein the test by addition of humic acid to liquid medium revealed significant effects. Namely, 0.3 g of humic acid was added to 2 liters of standard liquid medium (wherein commercial reagent of humic acid is available). Anti-HIV effects when this liquid medium was cultured in a 5-liter flask at 25° C. in a shake culture are shown hereafter.

*Effective dose for perfect HIV inhibition on the 3^{rd} day (the amount
of virus, 10TCID₅₀), co-cultured CD₄ cells and the HIV virus

| | | | Cell damage |
|---|---|---|---|
| Strains | Culture medium | Effective dose | inhibition |
| K-BIW | without humic acid | above 250 μl/ml | above 250 μl/ml |
| (cultured for | addition of humic | above 125 μl/ml | above 250 μl/ml |
| 70 days) | acid | | |

(In this case, ‘cell damage’ means differently from ‘cytotoxicity’, because constituents of Kabanoanatake are derived from natural objects and are taken orally.)

(7) Modification of Liquid Medium

It is possible that a liquid medium attenuates in a long-term culture (150 days, reciprocal shaking, 25° C.). To improve this, four test areas (A, B,C, D) were prepared beforehand by means of a 5 liters flask respectively, wherein 2 liters of standard medium were placed along with additional components, as follows:

A: Standard medium (without modification)

B: 0.3 g of humic acid was added to standard medium on the 61^{st} day after initiation of culture.

C: On the 76^{th} day after hyphae strains were placed into the standard medium, then one half of the medium that has the same composition as standard medium was added (to 1000 cc of water).

D: On the 61^{st} day after the incubation was initiated on the standard medium, whereto 0.3 g humic acid was placed, and furthermore on the 76^{th} day 0.3 g of humic acid and one half of the standard medium were added.

Anti-HIV activity (100% inhibition of syncytium formation) in each areas expressed by index were as follows; regarding A as 100, B is more than 200, C is more than 250, D is more than 300. The area modified culture medium was found to achieve a preferable high activity, compared to (A) the culture medium of which was not modified from the initiation of incubation. Describing it in detail, when the same carbon source was used, (A) showed as much as two times anti-HIV activity (per culture medium volume), as with the test case of humic acid described above. The test area (B) that one half of the carbon source (the same composition as the medium) was added at the time the carbon source was consumed showed as much as two point five times the perfect inhibition activity for HIV (per culture medium volume) as the standard medium. In the test area (D) where humic acid was combined with additional culture medium, the filtrate, as it is, caused cell damage (which does not indicate ‘cytotoxicity’) and made it unable to be measured. Consequently it was centrifuged to spin down to a solid content, which showed further high activity as (C) in the supernatant thereof.

(8) Culture in a Jar Fermenter

As an example of large-scale liquid culture method, large-scale culture of Kabanoanatake was cultivated using a 30-liter jar fermenter.

For the jar fermenter, MSJ-U2 (30-liter) was used (B. E. Marubishi, Co., Ltd). First, to prepare seed fungi, hyphae grown on PDA (potato dextrose agar) punched with No.4 cork borer were placed into 300 ml culture solution in three flasks. Here, a 500 ml shaking flask was used to leave more room for volume. Shaker rotated at 100 times/min., it was shake cultured at 25° C. for 31 days. Culture medium contained hyphae, which were equivalent to five shaking flasks in amount were placed on the jar fermenter. 20 liters of culture medium thereof had been incubated at 25° C., for 113 days, rotated 60 times/min, with air flow of 3 liters/min. In mid course, a small quantity of culture solution was added. The culture medium for the jar fermenter was composed as follows: 10 g of malt extract, 10 g of D-glucose, 3.0 g of polypeptone, 3.0 g of yeast extract, 0.1 liter of phosphate buffer solution (M 0.1, p H 5.0), and 0.9 liter of tap water, per Liter of medium. After completion of the culture, cultured hyphae extracted by hot water, anti-HIV activity thereof was measured. The hyphae culture solution was filtrated through gauze, to thus obtained hyphae, about as much as seven times the volume as H₂O was added, extracted at 90° C. for one hour by heating in hot water. The extracts were filtrated through gauze to remove hyphae, filtrated again through a paper filter, they are freeze-dried or refrigerated, ready to be used. Testing anti-HIV activity in the same method as described above, the effective dose thereof was determined to be 62.5 μl/ml, showing a high level of activity.

It follows from what has been said that it is capable of obtaining cultured products with strong anti-HIV activity or other high physiological activity over a long period, even when a carbon source or other nutrients in a medium become scarce in a liquid culture (a carbon source mainly using glucose, saccharose, malt sugar, etc.) or as well as in a solid culture (solid culture using sawdust or bagasse as the main ingredients), by adding the nutrients and activators. This has a significant meaning other than incubating for longer duration. According to the present invention, as stated previously on the basic investigations, the way was cleared to obtain the active ingredients in a short period of time, whereby industrial and economic culture methods for Kabanoanatake were shown. However, furthermore, the present invention revealed that long-term cultures are required for some strains to exert activity, depending on the breeds and the properties of Kabanoanatake hyphae, and the above improved culture methods has been established to be applied in such cases. Otherwise, in the case that harvest was not completed within regular hours (on time) according to convenience of cultivation work, the above improved culture methods have also established techniques to prevent the deterioration of cultured products and the decrease of activity, and to enhance the activity of the cultured products. To sum up, the present invention established industrial methods for short- and long-term cultures of Kabanoanatake.

The inventor took it into consideration that there have been increasingly tragic facts in the world concerning HIV infection after a person survives a sexual assault. If there is a method available to protect from infection by the HIV virus at least in case of an emergency, it would save women in a vulnerable position from the cruelty of HIV. Namely, in case that females that are compelled by force against their will, the inventor invented as a preventive agent or a safety sheath for body insertion in order to prevent HIV infection. Describing in detail, first, 0.1 g of extracted components of Kabanoanatake was mixed with 3 g of cocoa butter (Fuji OilCo,. Ltd.) as a vehicle. Cocoa butter was put in hot water to heat it up to at less than 40° C. and melted, whereto extracted components of Kabanoanatake are mixed and agitated. Lowering the temperature to 23° C., cocoa butter is made to crystallize. After that, the crystal is made to melt at 32° C., put in a mold, cooled to solidify, coated with a protective layer, in order to be a product. Anti-HIV activity was determined by the following procedure; this product (containing 0.1 g of effective dose) is inserted in the vagina after a certain length of time, the melted matter of item is wiped off by gauze of 6×4 cm, added distilled water, 10 cc, wrung for collecting the components, thereto added HIV using MT-4 cells, and in the vessel inhibition of growth of HIV was tested. The components being diluted artificially, the results were as follows.

| Lapse of time in insertion | Effective dose for HIV inhibition |
|---|---|
| 20 hrs. | 1000 μl/ml |
| 35 hrs. | 1000 μl/ml |
| 70 hrs. | 1000 μl/ml |

As evidenced by the results, even after being inserted in a body for 70 hours, the active ingredients exerted inhibitory effects against the growth of HIV. Thus, this suppository preventive agent shows great possibility of preventing HIV infection, by carrying this preventive item at an appropriate time when women must go to danger areas, or live there, if by any chance, such unexpected incidence should arise. Since the vehicle is made of cocoa butter, this preventive agent can be put into the mouth and is edible. This enables the prevention of oral infection with HIV. It is also available in the form of a soluble capsule.

In the similar forms such as solid material, soluble capsule, and other infusion of solutions, items containing Kabanoanatake extracts can be administered from the vagina or the anus, which may be used for preventing and treating HIV-related syndromes including cancer and bacterial disease. In particular, it is considered to be valid for the patients who are not able to take drugs because of disease.

Kabanoanatake extracts in the present invention, originally, can be taken orally, as medicine, either in the form of a powder or in the form of a liquid (ex. solution) and they can inhibit or prevent microbe-related syndromes caused by harmful pathogenic bacteria, a species of retroviruses, ATL (adult T-cell leukemia) and HIV may be inhibited or prevented. There is a case of a 50-year-old Japanese patient with ATL, who had taken Kabanoanatake extracts orally for two years and after that restored good health. Through adding and mixing these extracts in food and drink, it can be used on a daily basis, too. The concentration added as the standard is not limited to but is 0.1 to 10%. For instance, Kabanoanatake extracts of the present invention can be edible in the form of a powder, solution, or some other form of components of a substance, by adding and mixing them in seasonings such as soy sauce, bean paste, dressing, sauce, soup, and salt, etc. During production stages, confectionaries such as burns, cake, ice cream, chocolate, frozen dessert, rice cakes, wheat gluten, jelly, gummy, etc or foods such as sandwiches, noodles made of wheat flour, Japanese vermicelli, spaghetti, Chinese noodles, jam, batter and margarine could also be mixed with Kabanoanatake extracts (powder or liquid) to be useful in producing healthy foods. Likewise, during production or when drinking, Kabanoanatake extracts can be healthy drinks by mixing them with the following foods: meat-processed foods such as sausages, hamburg steaks, croquettes, tempra, steamed fish paste, seasoned meat, etc; alcohols or alcoholic beverages such as beer, wine, spirits, etc; garlic extract; juice of tomatoes, carrots, mixed vegetable, apple, grapes, etc; carbonated beverage such as cola; fermented tea such as black tea, oolong tea, yanrong tea, etc; yogurt beverages, coffee, green tea, cow milk and cocoa beverages. Furthermore, it is possible to make use of the active ingredients of Kabanoanatake in the present invention as folk medicine or health food, through using it as a mixture or concomitantly with Chinese herbal medicines or medicinal plants such as Cinnamomum sieboldi, Lithospermum erythrorhizon (roots), Lithospermum officinale (roots), Prunuspersica, Epimedium grandiflorum var thunbergianum, Carica papaya (enzyme), Ananas nanus (enzyme), Panax ginseng, Acanthopanax spinosus, Poria cocos, Ganoderma lucidum, Hipsizigus marmoreus, Ginkgo bloba (extract), Paeonia lactiflora, Cnidium officinale, Angelicae radix, etc. In the same way, as a medical treatment, it may be combined with anti-HIV agents such as AZT and DDI. The above foods added Kabanoanatake extracts which are exemplified by seasonings, confectionery, processed foods, meat and fish products, beverages are regarded as ‘health foods’ in a wide sense.

Artificially cultured Kabanoanatake, especially powder of sawdust cultures extracted by hot water, characterize a slightly bitter taste and a spicy savor. By adding this to beer and some other beverages, the flavor and bitter taste increase. It cannot only prevent the development of microbe-related syndromes, but can change the flavor of food. Based on this, the application will be developed that substitutes for hops, which is responsible for the bitterness of beer. Black pigment peculiar to Kabanoanatake may be utilized instead of succedaneum of caramel in producing soy sauce, and black powder therefrom is used as a safe and healthy edible dye. For example, the black powder from Kabanoanatake is utilized as a substances that enables the making of black steamed bean-jam bun which helps retaining health, and to increase the color depth of bean jam of red bean (Japanese azuki). Since this has no harmful effect on the human body, it can be applied to the use of natural dye for cloth, or hair dye, too. Furthermore, by mixing Kabanoanatake extracts in tobacco leaves during production stages, smokers are able to take anti-HIV and anti-bacterial substances orally.

What is claimed is:

1. The preventive and/or therapeutic agents containing extracts from Kabanoanatake as active ingredients and having preventive and therapeutic effects on various syndromes possibly caused by the microbes including HIV, a species of retroviruses, ALT (adult T-cell leukemia) and pathogenic bacteria.

2. The preventive and/or therapeutic agents as claimed in claim 1, wherein Kabanoanatake is a natural product.

3. The preventive and/or therapeutic agents as claimed in claim 1, wherein Kabanoanatake is an artificial-cultured product.

4. The preventive and/or therapeutic agents as claimed in claim 3, wherein Kabanoanatake is a sawdust-cultured product.

5. The preventive and/or therapeutic agents as claimed in claim 3, wherein Kabanoanatake is a liquid-cultured product.

6. The preventive and/or therapeutic agents as claimed in claim 1, wherein said microbe is the HIV virus.

7. The preventive and/or therapeutic agents as claimed in claim 6, wherein said agents have strong suppressive effect on syncytium formation caused by HIV and inhibitory effect on HIV infection at the concentration of 35 ng/ml.

8. The preventive and therapeutic agents as claimed in claim 6, wherein said agents have strong suppressive effect on syncytium formation caused by HIV and inhibitory effect on HIV infection at the concentration over 0.01 μg/ml.

9. The preventive and/or therapeutic agents as claimed in claim 1, wherein said microbe is pathogenic bacteria.

10. The preventive and/or therapeutic agents as claimed in claim 9, wherein said microbes are the bacteria selected from the group of enteropathogenic Escherichia coli, Klebsiella pneumoniae, Pseudomonas aeruginosa, MRSA Streptococcus, and Clostridium perfringens.

11. The preventive and/or therapeutic agents as claimed in claim 1, wherein said microbes are a retrovirus and ATL (adult T-cell leukemia) virus.

12. The preventive and/or therapeutic agents as claimed in claim 4, wherein said microbes is helicobacter pylori.

13. The method of liquid culture of Kabanoanatake, consisting of inoculating seed fungi from cultured Kabanoanatake in the liquid medium containing a mixture of a carbon source selected from the group of malt, glucose, saccharose and starch as well as peptone and yeast extract with water and phosphate buffer, incubating at 20 to 30° C. for 20 to 49 days and/or 100 days or longer and obtaining active ingredients in the hyphae and the medium.

14. The method as claimed in claim 13, wherein said active ingredients are contained in the medium composed of 10 g of malt extract, 10 g of glucose, 3 g of peptone and 3 g of yeast extract per Liter of the medium.

15. The method as claimed in claim 13, wherein humic acid is further added to said medium.

16. The method as claimed in claim 13, wherein sap of white birch is used instead of or in addition to water in said liquid medium.

17. The method as claimed in claim 13, wherein one or more wood constituents are added to said liquid medium.

18. The culture method as claimed in claim 13, wherein one or more wood constituents selected from lignin sulfonic acid, lignosulfonic acid sodium salt, lignosulfonic acid sodium salt acetate, lignin alkali, lignin organosolv, lignin organosolv acetate, 2-hydroxypropyl ether, lignin hydrolytic, hydroxymethyl derivative, lignin organosolv propionate, betulin, or lignin salts are added to said liquid medium.

19. The method as claimed in claim 17, wherein said wood constituents are used in the range of concentration between 0.00001% and 0.00075% (weight) for said liquid medium.

20. The method as claimed in claim 18, wherein said wood constituents are used at the concentration of 0.000293% (weight) for said liquid medium.

21. The method as claimed in claim 13, consisting of determining the time of full formation of said active ingredients based on color of the cultures, protein quantity in the culture medium, decrement of carbon source in the culture medium and pH of culture medium, and collecting said active ingredients.

22. The method as claimed in claim 13, wherein said shake culture is performed using devices such as ajar fermenter.

23. The method as claimed in claim 13, consisting of exposing growing hyphae to the light during culture.

24. The Healthy food effective on development of microbe-related syndromes, in which Kabanoanatake is a natural or an artificial culture, comprising mixing extracts of Kabanoanatake in the form of a powder or solution to: seasonings such as soy sauce, bean paste, dressing, sauce, soup, salt, etc; confectionaries such as burns, cake, ice cream, chocolate, frozen dessert, rice cakes, wheat gluten, jelly, gummy, etc; foods such as sandwiches, noodles made of wheat flour, Japanese vermicelli, spaghetti, Chinese noodles, potato powder, carrot leaf, jam, batter and margarine; meat-processed foods such as sausages, hamburg steaks, croquettes, tempra, steamed fish paste, seasoned meat, etc; alcohols or alcoholic beverages such as beer, wine, spirits, etc; garlic extract; juice of tomatoes, carrots, mixed vegetable, apple, grapes, etc; carbonated beverage such as cola; fermented tea such as black tea, oolong tea, yanrong tea, etc; yogurt beverages, coffee, green tea, cow milk and cocoa beverages.

25. The preventive and/or therapeutic agents as claimed in claim 1, 2, 3, 4, 5, 6, 7, 8, 9, 10 and 11, wherein said Kabanoanatake extracts are administered orally or from the anus or the vagina in the form of solid, fluid or liquid.

26. The preventive and/or therapeutic agents for HIV as claimed in claim 25, wherein said microbe is the HIV virus.

27. The preventive and/or therapeutic anti-microbial agents as claimed in claim 26, wherein said Kabanoanatake extracts are enabled to exert anti-microbe-related syndrome activity by using vehicles or solutions.
